(19) United States
(12) Patent Application Publication
(10) Pub. No.: US 2020/0199058 A1
(43) Pub. Date: Jun. 25, 2020

(54) SQUARYLIUM COMPOUNDS FOR USE IN DISPLAY DEVICES

(71) Applicant: NITTO DENKO CORPORATION, Osaka (JP)

(72) Inventors: Michael Welch, San Diego, CA (US); Shijun Zheng, San Diego, CA (US); Peng Wang, San Diego, CA (US); Ozair Siddiqui, Murrieta, CA (US); Wan-Yun Hsieh, San Diego, CA (US); Jie Cai, Oceanside, CA (US)

(21) Appl. No.: 16/620,600

(22) PCT Filed: Jun. 8, 2018
(86) PCT No.: PCT/US2018/036741

§ 371 Date: Dec. 9, 2019

Related U.S. Application Data
(60) Provisional application No. 62/517,722, filed on Jun. 9, 2017.

(51) Int. Cl. C07C 49/835; C09B 57/00; G02B 5/22; G02B 1/111

(52) U.S. Cl. CPC C07C 49/835; G02B 1/111; G02B 5/223; C09B 57/007

(57) ABSTRACT

Optionally substituted squarylium compounds, such as those depicted in Formula 1, may be useful in filters for display devices.

### BACKGROUND

### Field

The embodiments include compounds for use in color filters through which light passes.

### Description of the Related Art

In color reproduction, the color gamut can be a given complete subset of colors. The most common usage refers to the subset of colors which can be accurately represented in a given circumstance, such as by a certain output device. For example, the wide-gamut Red Green Blue (RGB) color space (or Adobe Wide Gamut RGB) is an RGB color space developed by Adobe Systems that offers a large gamut by using pure spectral primary colors. It is asserted to be able to store a wider range of color values than sRGB or Adobe RGB color spaces. So, it is believed, that a display device which could provide a wider gamut could enable the device to portray more vibrant colors.

### SUMMARY

Some embodiments include a squarylium compound represented by Formula 1:

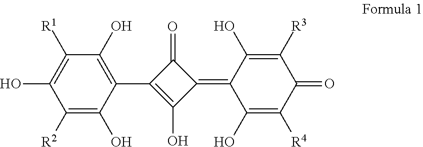

or a tautomer thereof;
wherein R¹, R², R³, and R⁴ are independently H or a substituent such as L, —CO-L, Ar, or -L-Ar.

Some embodiments include an optical filter comprising: a squarylium compound, such as a compound of Formula 1; and a polymer matrix, wherein the squarylium compound is disposed within the polymer matrix; wherein the optical filter has a quantum yield of less than about 1%.

Some embodiments include a display device comprising the optical filter described herein and an RBG source positioned to allow viewing of the RGB source through the optical filter.

### BRIEF DESCRIPTION OF THE DRAWINGS

FIG. 1 is a schematic diagram of an example of a display device having a filter comprising the compound described herein.

FIG. 2 is a graph depicting the normalized absorption spectra of a film comprising Squarylium Compound 14.

### DETAILED DESCRIPTION

One problem with a wide color gamut is that the green and red colors can be spectrally adjacent to each other and not fully distinguishable from each other. One way to reduce these color aberrations can be to utilize an absorbing dye to reduce the amount of spectral emission and overlap in this region. In some cases, wavelength converting materials can be incorporated into display device filters. In some cases, an absorbing dye having an absorption wavelength between about 580 nm to about 620 could be useful. In addition, to reduce the effect of the removal of emitted light while sharpening the distinction between the perceived green and red colors, a narrow absorption spectrum, as indicated by a narrow full width half maximum (FWHM) can be desirable.

The squaraines are a class of near-IR dyes. They can be useful in conjunction with color displays, wherein the dye can be useful as a sharp minimum value absorption filter in the wavelength region of 560 to 620 nm However, there are several potential problems with these compounds.

One problem is that strong nucleophiles can attack the electron-deficient cyclobutene ring which can lead to a loss of the dye's blue color. Another potential problem is that squaraines dyes tend to form aggregates, which can lead to a substantial broadening of their absorption bands. A possible solution to these problems can be to encapsulate the dyes inside a protective molecular container or framework, such as encapsulating the dye as a rotaxane, to protect it from nucleophiles. However, these compounds are difficult to make.

By employing a newly designed molecular structure, an example shown below, we report a new material that can be used in filters and/or display device applications. The squarylium compounds described herein can effectively and selectively absorb light in the region 570 to 610 nm, between a green color and a red color with a particularly narrow half-value width so that they may aid in the distinction between perceived green or red colors. Therefore, they can be particularly useful dyes for color correction, improving color purity or broadening the color reproduction range.

Some filters comprising a squarylium compound described herein can have a reduced fluorescence, e.g., display a reduced quantum yield, such as less than about 10% (or 0.1), less than about 6% (or 0.06), less than about 5% (or 0.05), less than about 4% (or 0.04), less than about 3% (or 0.03), less than about 2% (or 0.02), less than about 1% (or 0.01), less than about 0.8% (or 0.008), less than about 0.75 (0.0075), less than about 0.7% (or 0.007), less than 0.65% (0.0065), less than about 0.5% (or 0.005), less than about 0.45% (0.0045), less than about 0.4% (or 0.004), or less than about 0.3% (or 0.003). Therefore, they can be particularly useful dyes for display device color correction, improving color purity, or broadening the color reproduction range. Quantum yield measurements in solution can be made by comparing the integrated fluorescence emission of the squarylium compound described herein, with the integrated fluorescence of Nile blue A (QY=0.23 in ethanol) at equal dye absorbance, at the excitation wavelength. The fluorescence of the buffer alone is subtracted from that of the sample for each measurement. Quantum yield in a film can also be determined using a quantum yield spectrophotometer, e.g., Quantaurus-QY spectrophotometer (Hamamatsu, Inc., Campbell, Calif., USA). In some embodiments, the squarylium compounds described herein can be weakly fluorescent or essentially non-fluorescent.

The squarylium compounds of following formula can be compounds which effectively and selectively absorb light in the region above about 550 nm, about 500-600 nm, about 570-610 nm, about 550-630 nm, about 560-570 nm, about 565-570 nm, about 570-580 nm, about 570-575 nm, about 575-580 nm, about 580-585 nm, about 585-590 nm, about 580-590 nm, about 560-620 nm, about 565-615 nm, about 580-600 nm, or about 580-620 nm. Ranges that encompass the following peak absorptions are of particular interest: about 568 nm, about 575 nm, about 578 nm, about 579 nm, about 580 nm, about 581 nm, about 582 nm, about 583 nm, about 584 nm, and about 588 nm.

In some embodiments, a shoulder in absorption spectra, e.g., about 475 nm in FIG. 2, can be removed and/or reduced by modifying the compound's chemical structure[s] to be more rigid, thus restricting rotations which may cause vibronic features in absorption, which can be reflected in the spectra as a shoulder.

For some uses, such as helping to distinguish between green and/or red colors, the squarylium compounds can have a particularly narrow full width at half maximum, such as about 60 nm or less, about 50 nm or less, about 45 nm or less, about 40 nm or less, about 35 nm or less, about 35-60 nm, about 35-40 nm, about 40-50 nm, about 50-60 nm, or any full width at half maximum in a range bounded by any of these values.

An optical filter described herein typically contains a squarylium compound dispersed within a polymer matrix.

Unless otherwise indicated, when a compound or chemical structural feature such as aryl is referred to as being “optionally substituted,” it includes a feature that has no substituents (i.e. unsubstituted), or a feature that is “substituted,” meaning that the feature has one or more substituents. The term “substituent” has the broadest meaning known to one of ordinary skill in the art, and includes a moiety that occupies a position normally occupied by one or more hydrogen atoms attached to a parent compound or structural feature. In some embodiments, a substituent may be an ordinary organic moiety known in the art, which may have a molecular weight (e.g. the sum of the atomic masses of the atoms of the substituent) of 15-50 g/mol, 15-100 g/mol, 15-200 g/mol, or 15-500 g/mol. Some substituents include C₁₋₁₂H₃₋₂₅, optionally substituted phenyl, C₁₋₁₃ hydrocarbyl, optionally substituted C₁₋₁₃—CO-hydrocarbyl, optionally substituted —CH₂-phenyl, etc.

For convenience, the term “molecular weight” is used with respect to a moiety or part of a molecule to indicate the sum of the atomic masses of the atoms in the moiety or part of a molecule, even though it may not be a complete molecule.

In some embodiments, the phenyl and/or benzyl may have 0, 1, 2, 3, or 4 substituents independently selected from: R′, —OR′, —COR′, —CO₂R′, —OCOR′, —NR′COR″, CONR′R″, —NR′R″, F; Cl; Br; I; nitro; CN, etc., wherein R′ and R″ are independently H, optionally substituted phenyl, or C₁₋₆ alkyl, such as methyl, ethyl, propyl, isomers, cyclopropyl, butyl isomers, cyclobutyl isomers (such as cyclobutyl, methylcyclopropyl, etc.), pentyl isomers, cyclopentyl isomers, hexyl isomers, cyclohexyl isomers, etc. In some embodiments, the substitutents can be —OH.

A squarylium compound may have the structure depicted in Formula 1.

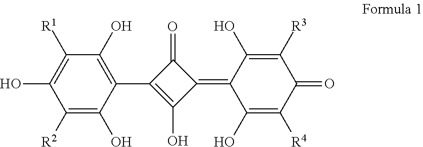

or a tautomer thereof; wherein R¹, R², R³, and R⁴ are independently H, L, —CO-L, Ar, or -L-Ar.

Any reference a compound herein by structure of formula includes any tautomers of the compounds represented. For example, depending the symmetry, a compound of Formula 1 can be rapidly converted to a tautomer represented by Formula 1T. Other tautomers may also be possible. However, for convenience, only one of the tautomeric forms is typically identified herein.

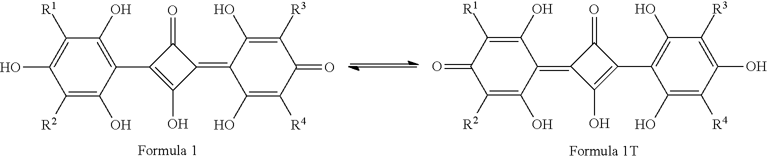

With respect to any relevant structural representation, such as Formula 1, in some embodiments R¹ may be H, or any suitable substituent, such as L, —CO-L, Ar, or -L-Ar. In some embodiments, R¹ is a bulky substituent. In some embodiments, R¹ is L. In some embodiments, R¹ is —CO-L. In some embodiments, R¹ is Ar. In some embodiments, R¹ is -L-Ar. In some embodiments, R¹ may be any suitable substituent, such as, C₁₋₁₂ alkyl, such as CH₃, C₂alkyl (e.g. CH₂CH₃), C₃ alkyl (e.g. CH₂CH₂CH₃, CHCH₃CH₃etc.), C₄ alkyl, C₅ alkyl, or C₆ alkyl; C₂₋₆alkenyl, such as C₂ alkenyl (e.g. CH═CH), C₃ alkenyl (e.g. CH₂—CH═CH₂, etc.), C₄ alkenyl, C₅ alkenyl, or C₆ alkenyl; optionally substituted phenyl (e.g. C₆H₃(OH)₂); C₁₋₁₃—CO-hydrocarbyl, such as C₁₋₁₃—CO-alkyl, e.g. —COCH₃, —COCH₂CH₃, etc.; optionally substituted —CH₂-phenyl (e.g. —CH₂—C₆H₅, —CH₂—C₆H₃(C(CH₃)₂)₂ etc.); or optionally substituted —CH₂CH═CH-phenyl. In some embodiments, R¹ is H. In some embodiments, R¹ is linear C₁₋₄ alkyl. In some embodiments, R¹ is linear C₃₋₄ alkenyl. In some embodiments, R¹ is 3,5-dihydroxyphenyl. In some embodiments, R¹ is 3,5-di(tert-butyl)phenylmethyl. In some embodiments, R¹ is —CH₂CH₂CH₃. In some embodiments, R¹ is n-butyl. In some embodiments, R¹ is t-butyl. In some embodiments, R¹ is —CH₂CH═CH₂. In some embodiments, R¹ is COCH₃. In some embodiments, R¹ is benzyl. In some embodiments, R¹ is —CH₂C═CH-phenyl.

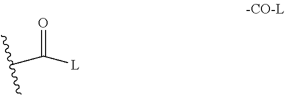

With respect to any relevant structural representation, such as Formula 1, in some embodiments the Ar of R¹ is:

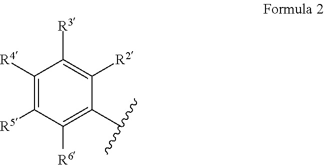

With respect to any relevant structural representation, such as Formula 2, R^{2′} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{2′} is H.

With respect to any relevant structural representation, such as Formula 2, R^{3′} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{3′} is H. In some embodiments, R^{3′} is —C(CH₃)₃. In some embodiments, R^{3′} is OH.

With respect to any relevant structural representation, such as Formula 2, R^{4′} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{4′} is H.

With respect to any relevant structural representation, such as Formula 2, R^{5′} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{5′} is H. In some embodiments, R^{5′} is —C(CH₃)₃. In some embodiments, R^{5′} is OH.

With respect to any relevant structural representation, such as Formula 2, R^{6′} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{6′} is H.

With respect to any relevant structural representation, such as Formula 1, in some embodiments R² may be H, or any suitable substituent, such as L, —CO-L, Ar, or -L-Ar. In some embodiments, R² is a bulky substituent. In some embodiments, R² is L. In some embodiments, R² is —CO-L. In some embodiments, R² is Ar. In some embodiments, R² is -L-Ar. In some embodiments, R² may be any suitable substituent, such as, C₁₋₁₂ alkyl, such as CH₃, C₂alkyl (e.g. CH₂CH₃), C₃ alkyl (e.g. CH₂CH₂CH₃, CHCH₃CH₃ etc.), C₄ alkyl, C₅ alkyl, or C₆ alkyl; C₂₋₆alkenyl, such as C₂ alkenyl (e.g. CH═CH), C₃ alkenyl (e.g. CH₂—CH═CH₂, etc.), C₄ alkenyl, C₅ alkenyl, or C₆ alkenyl; optionally substituted phenyl (e.g. C₆H₃(OH)₂); C₁₋₁₃—CO-hydrocarbyl, such as C₁₋₁₃—CO-alkyl, e.g. —COCH₃, —COCH₂CH₃, etc.; optionally substituted —CH₂-phenyl (e.g. —CH₂—C₆H₅, —CH₂—C₆H₃(C(CH₃)₂)₂ etc.); or optionally substituted —CH₂CH═CH-phenyl. In some embodiments, R² is linear C₁₋₄ alkyl. In some embodiments, R² is linear C₃₋₄ alkenyl. In some embodiments, R² is H. In some embodiments, R² is 3,5-dihydroxyphenyl. In some embodiments, R² is 3,5-di(tert-butyl)phenylmethyl. In some embodiments, R² is —CH₂CH₂CH₃. In some embodiments, R² is n-butyl. In some embodiments, R² is t-butyl. In some embodiments, R² is —CH₂CH═CH₂. In some embodiments, R² is COCH₃. In some embodiments, R² is benzyl. In some embodiments, R² is —CH₂C═CH-phenyl. In some embodiments, R³ is —CO-L, Ar, or -L-Ar. In some embodiments, R³ is CH₃. In some embodiments, R³ is C₃ alkyl. In some embodiments, R³ is CH₂CH₃, acyclic C₄₋₆ alkyl, or an acyclic C₁₋₆ hydrocarbyl that is not alkyl. In some embodiments, R³ is an acyclic C₄₋₆ alkyl, an acyclic C₂₋₆ hydrocarbyl that is not alkyl, —CO-L, Ar, or -L-Ar.

With respect to any relevant structural representation, such as Formula 1, in some embodiments the Ar of R² is:

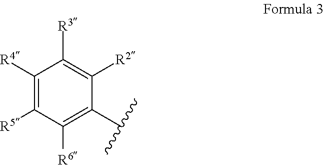

With respect to any relevant structural representation, such as Formula 3, R^{2″} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{2″} is H.

With respect to any relevant structural representation, such as Formula 3, R^{3″} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{3″} is H. In some embodiments, R^{3″} is —C(CH₃)₃. In some embodiments, R^{3″} is OH.

With respect to any relevant structural representation, such as Formula 3, R^{4″} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{4″} is H.

With respect to any relevant structural representation, such as Formula 3, R^{5″} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{5″} is H. In some embodiments, R^{5″} is —C(CH₃)₃. In some embodiments, R^{5″} is OH.

With respect to any relevant structural representation, such as Formula 3, R^{6″} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{6″} is H.

With respect to any relevant structural representation, such as Formula 1, in some embodiments R³ may be H, or any suitable substituent, such as L, —CO-L, Ar, or -L-Ar. In some embodiments, R³ is a bulky substituent. In some embodiments, R³ is L. In some embodiments, R³ is —CO-L. In some embodiments, R³ is Ar. In some embodiments, R³ is -L-Ar. In some embodiments, R³ may be any suitable substituent, such as, C₁₋₁₂ alkyl, such as CH₃, C₂alkyl (e.g. CH₂CH₃), C₃ alkyl (e.g. CH₂CH₂CH₃, CHCH₃CH₃etc.), C₄ alkyl, C₅ alkyl, or C₆ alkyl; C₂₋₆ alkenyl, such as C2 alkenyl (e.g. CH═CH), C₃ alkenyl (e.g. CH₂—CH═CH₂, etc.), C₄ alkenyl, C₅ alkenyl, or C₆ alkenyl; optionally substituted phenyl (e.g. C₆H₃(OH)₂); C₁₋₁₃—CO-hydrocarbyl, such as C₁₋₁₃—CO-alkyl, e.g. —COCH₃, —COCH₂CH₃, etc.; optionally substituted —CH₂-phenyl (e.g. —CH₂—C₆H₅, —CH₂—C₆H₃(C(CH₃)₂)₂ etc.); or optionally substituted —CH₂CH═CH-phenyl. In some embodiments, R³ is linear C₁₋₄ alkyl. In some embodiments, R³ is linear C₃₋₄ alkenyl. In some embodiments, R³ is H. In some embodiments, R³ is 3,5-dihydroxyphenyl. In some embodiments, R³ is 3,5-di(tert-butyl)phenylmethyl. In some embodiments, R³ is —CH₂CH₂CH₃. In some embodiments, R³ is n-butyl. In some embodiments, R³ is t-butyl. In some embodiments, R³ is —CH₂CH═CH₂. In some embodiments, R³ is COCH₃. In some embodiments, R³ is benzyl. In some embodiments, R³ is —CH₂C═CH-phenyl

With respect to any relevant structural representation, such as Formula 1, in some embodiments the Ar of R³ is:

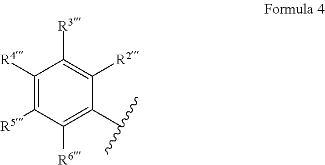

With respect to any relevant structural representation, such as Formula 4, R^{2′″} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{2′″} is H.

With respect to any relevant structural representation, such as Formula 4, R^{3′″} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{3′″} is H. In some embodiments, R^{3′″} is —C(CH₃)₃. In some embodiments, R^{3′″} is OH.

With respect to any relevant structural representation, such as Formula 4, R^{4′″} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{4′″} is H.

With respect to any relevant structural representation, such as Formula 4, R^{5′″} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{5′″} is H. In some embodiments, R^{5′″} is —C(CH₃)₃. In some embodiments, R^{5′″} is OH.

With respect to any relevant structural representation, such as Formula 4, R^{6′″} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{6′″} is H.

With respect to any relevant structural representation, such as Formula 1, in some embodiments R⁴ may be H, or any suitable substituent, such as L, —CO-L, Ar, or -L-Ar. In some embodiments, R⁴ is a bulky substituent. In some embodiments, R⁴ is L. In some embodiments, R⁴ is —CO-L. In some embodiments, R⁴ is Ar. In some embodiments, R⁴ is -L-Ar. In some embodiments, R⁴ may be any suitable substituent, such as, C₁₋₁₂ alkyl, such as CH₃, C₂alkyl (e.g. CH₂CH₃), C₃ alkyl (e.g. CH₂CH₂CH₃, CHCH₃CH₃etc.), C₄ alkyl, C₅ alkyl, or C₆ alkyl; C₂₋₆ alkenyl, such as C₂ alkenyl (e.g. CH═CH), C₃ alkenyl (e.g. CH₂—CH═CH₂, etc.), C₄ alkenyl, C₅ alkenyl, or C₆ alkenyl; optionally substituted phenyl (e.g. C₆H₃(OH)₂); C₁₋₁₃—CO-hydrocarbyl, such as C₁₋₁₃—CO-alkyl, e.g. —COCH₃, —COCH₂CH₃, etc.; optionally substituted —CH₂-phenyl (e.g. —CH₂—C₆H₅, —CH₂—C₆H₃(C(CH₃)₂)₂ etc.); or optionally substituted —CH₂CH═CH-phenyl. In some embodiments, R⁴ is linear C₁₋₄ alkyl. In some embodiments, R⁴ is linear C₃₋₄ alkenyl. In some embodiments, R⁴ is H. In some embodiments, R⁴ is 3,5-dihydroxyphenyl. In some embodiments, R⁴ is 3,5-di(tert-butyl)phenylmethyl. In some embodiments, R⁴ is —CH₂CH₂CH₃. In some embodiments, R⁴ is n-butyl. In some embodiments, R⁴ is t-butyl. In some embodiments, R⁴ is —CH₂CH═CH₂. In some embodiments, R⁴ is COCH₃. In some embodiments, R⁴ is benzyl. In some embodiments, R⁴ is —CH₂C═CH-phenyl. In some embodiments, R⁴ is L, —CO-L, Ar, or -L-Ar. In some embodiments, R⁴ is H, L, —CO-L, Ar, or -L-Ar. In some embodiments, R⁴ is H, an acyclic C₂₋₆ hydrocarbyl, —CO-L, Ar, or -L-Ar. In some embodiments, R⁴ is H, C₁₋₂ alkyl, an acyclic C₄₋₆ alkyl, an acyclic C₂₋₆ hydrocarbyl that is not alkyl, —CO-L, Ar, or -L-Ar. In some embodiments, R⁴ is an acyclic C₄₋₆ alkyl, an acyclic C₂₋₆ hydrocarbyl that is not alkyl, —CO-L, Ar, or -L-Ar.

With respect to any relevant structural representation, such as Formula 1, in some embodiments the Ar of R⁴ is:

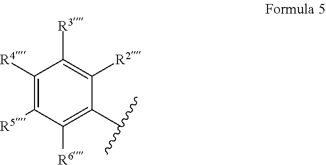

With respect to any relevant structural representation, such as Formula 5, R^{2″″} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{2″″} is H.

With respect to any relevant structural representation, such as Formula 5, R^{3″″} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{3″″} is H. In some embodiments, R^{3″″} is —C(CH₃)₃. In some embodiments, R^{3″″} is OH.

With respect to any relevant structural representation, such as Formula 5, R^{4″″} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{4″″} is H.

With respect to any relevant structural representation, such as Formula 5, R^{5″″} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{5″″} is H. In some embodiments, R^{5″″} is —C(CH₃)₃. In some embodiments, R^{5″″} is OH.

With respect to any relevant structural representation, such as Formula 5, R^{6″″} is H or any suitable substituent, such as C₁₋₆ alkyl (e.g. CH₃, C₂ alkyl, C₃ alkyl, C₄ alkyl, etc.), C₂₋₆ alkenyl (e.g. C₂ alkenyl, C₃ alkenyl, C₄ alkenyl, etc.) COH, OH, etc. In some embodiments, R^{6″″} is H.

With respect to R¹, R², R³, and R⁴, each L is independently an acyclic C₁₋₆ hydrocarbon group, such as C₁₋₆ alkane (e.g. CH₃, CH₂CH₃, CH₂CH₂CH₃, etc.) or C₂₋₆ alkene (e.g. CH₂═CH₂, CH═CH—CH₃, CH₂—CH═CH—CH₃, etc.). In some embodiments, R¹, R², R³, and R⁴ are independently CO-L, such as C═O—CH₃, C═O—CH₂—CH₃, etc.

With respect to R¹, R², R³, and R⁴, each Ar is independently optionally substituted C₆₋₁₀ aryl group, such as optionally substituted phenyl, such as —C₆H₃(OH)₂ or —C₆H₃(C(CH₃)₃)₂.

In some embodiments, all substituents of each Ar are represented by the empirical formula C₁₋₁₀H₃₋₂₁O₀₋₁ (e.g. C₄H₉ such as —C(CH₃)₃). In some embodiments, R¹, R², R³, and R⁴ are independently -L-Ar, such as, —CH₂—Ar (e.g. CH₂—C₆H₅, etc.) or CH₂CH═CH—Ar (e.g. CH₂CH═CH—C₆H₅, etc.)

In some embodiments, the squarylium compound is not:

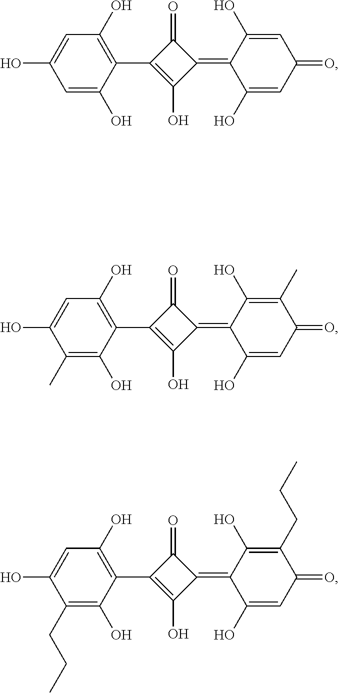 [SMILES: O=C1C=C(O)C(=C2C(=O)C(c3c(O)cc(O)cc3O)=C2O)C(O)=C1 ; CC1=C(O)/C(=C2\C(=O)C(c3c(O)cc(O)c(C)c3O)=C2O)C(O)=CC1=O ; CCCC1=C(O)/C(=C2\C(=O)C(c3c(O)cc(O)c(CCC)c3O)=C2O)C(O)=CC1=O]

or a tautomer thereof.

With respect to any relevant structural representation, such as Formula 1, in some embodiments, R² is H, and R⁴ is L, —CO-L, Ar, or -L-Ar. In some embodiments, R² is CH₂CH₃, acyclic C₄₋₆ alkyl, or an acyclic C₁₋₆ hydrocarbyl that is not alkyl, and R⁴ is H, L, —CO-L, Ar, or -L-Ar. In some embodiments, R² is CH₃ and R⁴ is H, an acyclic C₂₋₆ hydrocarbyl, —CO-L, Ar, or -L-Ar. In some embodiments, R² is C₃ alkyl and R⁴ is H, C₁₋₂ alkyl, an acyclic C₄₋₆ alkyl, an acyclic C₂₋₆ hydrocarbyl that is not alkyl, —CO-L, Ar, or -L-Ar. In some embodiments, R² and R⁴ are different. In some embodiments, R² and R⁴ are independently an acyclic C₄₋₆ alkyl, an acyclic C₂₋₆ hydrocarbyl that is not alkyl, —CO-L, Ar, or -L-Ar.

Some embodiments include a compound depicted below. Each of these compounds may be optionally substituted.

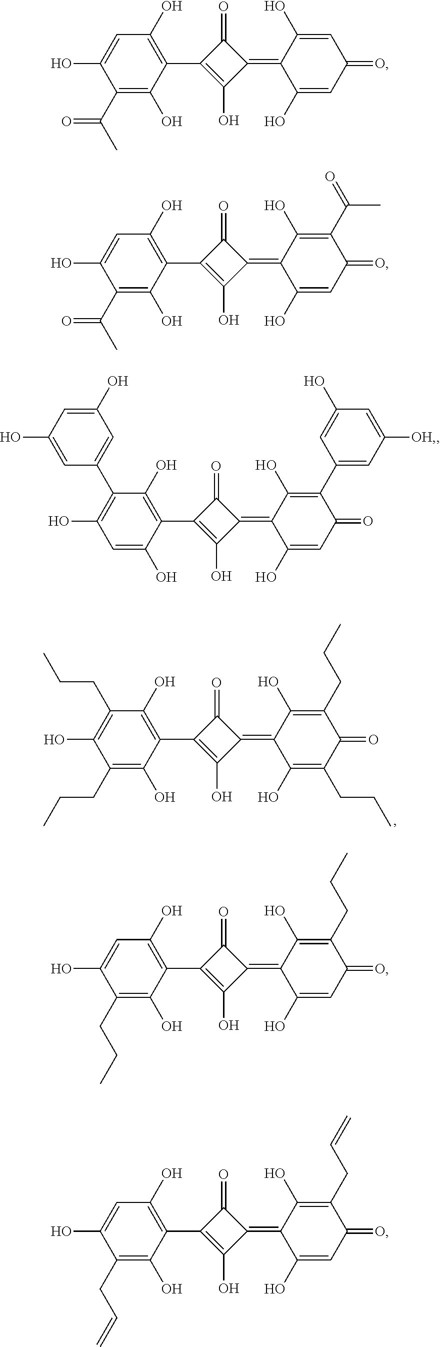

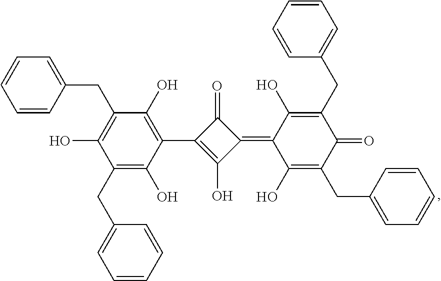 [SMILES: O=C1C(Cc2ccccc2)=C(O)C(=C2C(=O)C(c3c(O)c(Cc4ccccc4)c(O)c(Cc4ccccc4)c3O)=C2O)C(O)=C1Cc1ccccc1]

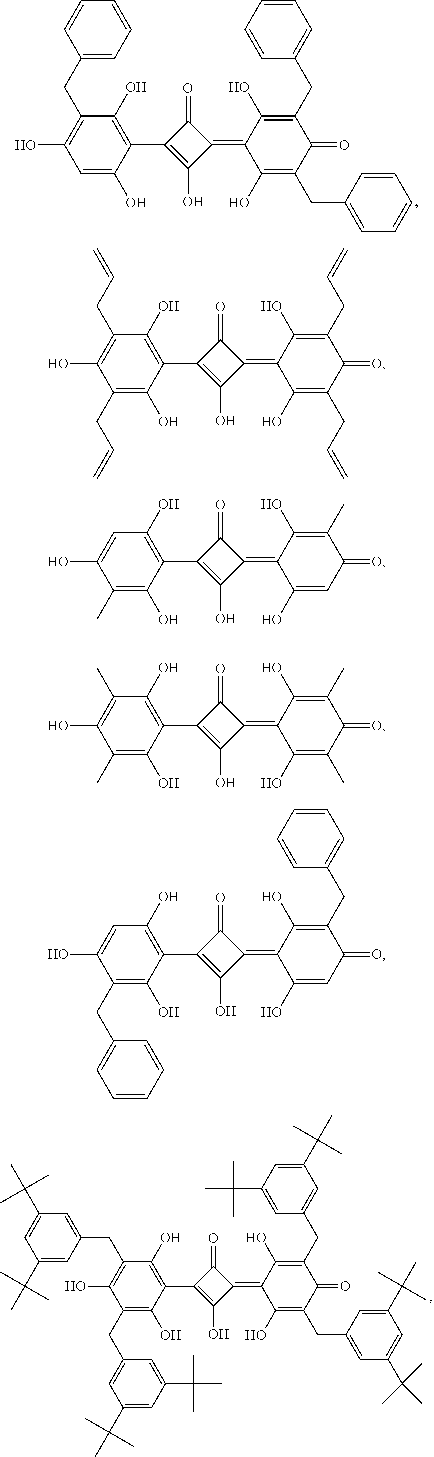

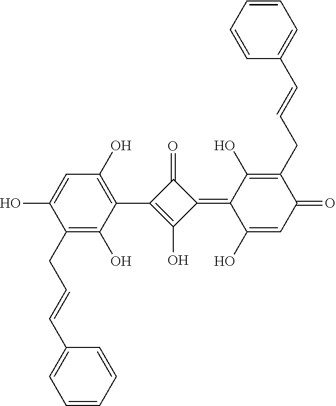 [SMILES: O=C1C=C(O)/C(=C2/C(=O)C(c3c(O)cc(O)c(C/C=C/c4ccccc4)c3O)=C2O)C(O)=C1C/C=C/c1ccccc1]

The polymer matrix may be composed of, or may comprise, any suitable polymer, such as an acrylic, a polycarbonate, an ethylene-vinyl alcohol copolymer, an ethylene-vinyl acetate copolymer or a saponification product thereof, an AS, a polyester, a vinyl chloride-vinyl acetate copolymer, a polyvinyl butyral, polyvinylphosphonic acid (PVPA), a polystyrene, a phenolic resin, a phenoxy resin, a polysulfone, a nylon, a cellulosic resin, a cellulose acetate, etc. In some embodiments, the polymer is an acrylic or acrylate polymer. In some embodiments, the polymer matrix comprises poly(methyl methacrylate). The polymer may act as a binder resin.

An oxygen scavenging agent may be present in the polymer matrix to, e.g. help reduce oxidation of the coordination complex. This may help to improve the color stability of the filter.

The filter may have any suitable configuration where the squarylium compound is dispersed within a polymer matrix. In some embodiments, the polymer matrix acts as a binder resin. Representative examples of the configuration of the filter include a laminate structure composed of a transparent sheet or film substrate and a layer containing the compound dispersed within a polymer that acts as a binder resin, and a single layer structure, e.g., a sheet or film made of a binder resin containing the compound.

In some embodiments, the polymer matrix containing the squarylium compound is in the form of a layer having a thickness of about 0.1-100 μm, about 0.1-20 μm, about 20-40 μm, about 40-60 μm, about 60-100 μm, about 0.1 um to about 50 μm, or about 30 μm to about 100 μm.

If two or more squarylium compounds are used, they can be mixed into a single layer or a single film of the above laminate, or a plurality of layers or films each containing a compound may be provided. In such a case, a laminate is formed even in the above-described latter case. Filter properties may be tuned by adjusting the binder resins depending on the respective squarylium compound used in the resin.

The laminate filter can be prepared by, for example, (1) a method comprising dissolving or dispersing the compound and a binder resin in an appropriate solvent and applying the solution or dispersion on a transparent sheet or film substrate by a conventional method, followed by drying, (2) a method comprising melt-kneading the compound and a binder resin, molding the mixture into a film or a sheet by a conventional molding technique for thermoplastic resins such as extrusion, injection molding or compression molding, and adhering the film or sheet to a transparent substrate, e.g., with an adhesive, (3) a method comprising extrusion laminating a molten mixture of the squarylium compound and a binder resin on a transparent substrate, (4) a method comprising co-extruding a molten mixture of the squarylium compound and a binder resin with a molten resin for a transparent substrate, or (5) a method comprising molding a binder resin into a film or a sheet by extrusion, injection molding, compression molding, etc., bringing the film or the sheet into contact with a solution of the squarylium compound, and the thus dyed film or sheet is adhered to a transparent substrate, e.g., with an adhesive.

The single layer sheet or film comprising a resin containing the squarylium compound is prepared by, for example, (1) a method comprising casting a solution or dispersion of the squarylium compound and a binder resin in an appropriate solvent on a carrier followed by drying, (2) a method comprising melt-kneading the squarylium compound and a binder resin and molding the mixture into a film or a sheet by a conventional molding technique for thermoplastic resins such as extrusion, injection molding or compression molding, or (3) a method comprising molding a binder resin into a film or a sheet by extrusion, injection molding, compression molding, etc. and bringing the film or the sheet into contact with a solution of the squarylium compound.

The laminate filter can comprise a transparent substrate having a squarylium compound-containing resin layer disposed on the surface of the transparent substrate. The squarylium compound-containing resin layer may comprise a binder resin and the squarylium compound dispersed within the binder resin. This type of laminate filter may be produced by coating a transparent sheet or film substrate with a coating composition prepared by dissolving the squarylium compound and a binder resin in an appropriate solvent or dispersing the particles of the compound having a particle size of 0.1 to 3 micrometers (um) and a binder resin in a solvent and drying the coating film.

The method of making the filter can be chosen according to the layer structure and material fit for a particular use.

Materials of the transparent substrate which can be used in the filter for LCD's and/or PDPs are not particularly limited as far as they are substantially transparent, having little light absorption, and causing little light scattering. Examples of suitable materials include glass, polyolefin resins, amorphous polyolefin resins, polyester resins, polycarbonate resins, acrylic resins, polystyrene resins, polyvinyl chloride resins, polyvinyl acetate resins, polyarylate resins, and polyether sulfone resins. A suitable example includes poly (methyl methacrylate) (PMMA).

The resin can be molded into a film or a sheet by conventional molding methods, such as injection molding, T-die extrusion, calendering and compression molding, and/or by casting a solution of the resin in an organic solvent. The resin can contain commonly known additives, such as anti-heat aging agents, lubricants, scavenging agents, and antioxidants. The substrate can have a thickness of 10 micrometers (μm) to 5 mm. The resin film or sheet may be an unstretched or stretched film or sheet. The substrate may be a laminate of the above-described material and other films or sheets.

If desired, the transparent substrate can be subjected to a known surface treatment, such as a corona discharge treatment, a flame treatment, a plasma treatment, a glow discharge treatment, a surface roughening treatment, or a chemical treatment. If desired, the substrate can be coated with an anchoring agent or a primer.

The solvent which can be used for dissolving or dispersing the dye and the resin can include alkanes, such as butane, pentane, hexane, heptane, and octane; cycloalkanes, such as cyclopentane, cyclohexane, cycloheptane, and cyclooctane; alcohols, such as ethanol, propanol, butanol, amyl alcohol, hexanol, heptanol, octanol, decanol, undecanol, diacetone alcohol, and furfuryl alcohol; cellosolves, such as methyl cellosolve, ethyl cellosolve, butyl cellosolve, methyl cellosolve acetate, and ethyl cellosolve acetate; propylene glycol and its derivatives, such as propylene glycol monomethyl ether, propylene glycol monoethyl ether, propylene glycol monobutyl ether, propylene glycol monomethyl ether acetate, propylene glycol monoethyl ether acetate, propylene glycol monobutyl ether acetate, and dipropylene glycol dimethyl ether; ketones, such as acetone, methyl amyl ketone, cyclohexanone, and acetophenone; ethers, such as dioxane and tetrahydrofuran; esters, such as butyl acetate, amyl acetate, ethyl butyrate, butyl butyrate, diethyl oxalate, ethyl pyruvate, ethyl 2-hydroxybutyrate, ethyl acetoacetate, methyl lactate, ethyl lactate, and methyl 3-methoxypropionate; halogenated hydrocarbons, such as chloroform, methylene chloride, and tetrachloroethane; aromatic hydrocarbons, such as benzene, toluene, xylene, and cresol; and highly polar solvents, such as dimethyl formamide, dimethyl acetamide, and N-methylpyrrolidone.

An RGB source is a light source which emits at the same time red, green and blue light. Such sources are required mainly for color display applications. A wide range of colors can be obtained by mixing different amounts of red, green and blue light (additive color mixing). Suitable RGB sources include, but are not limited to, a cathode ray tube (CRT), liquid crystal display (LCD), plasma display, or organic light emitting diode (OLED) display such as a television, a computer monitor, or a large scale screen. Each pixel on the screen can be built by driving three small and very close but still separated RGB light sources. At common viewing distance, the separate sources may seem indistinguishable, which can trick the eye to see a given solid color. All the pixels arranged together in the rectangular screen surface conforms the color image.

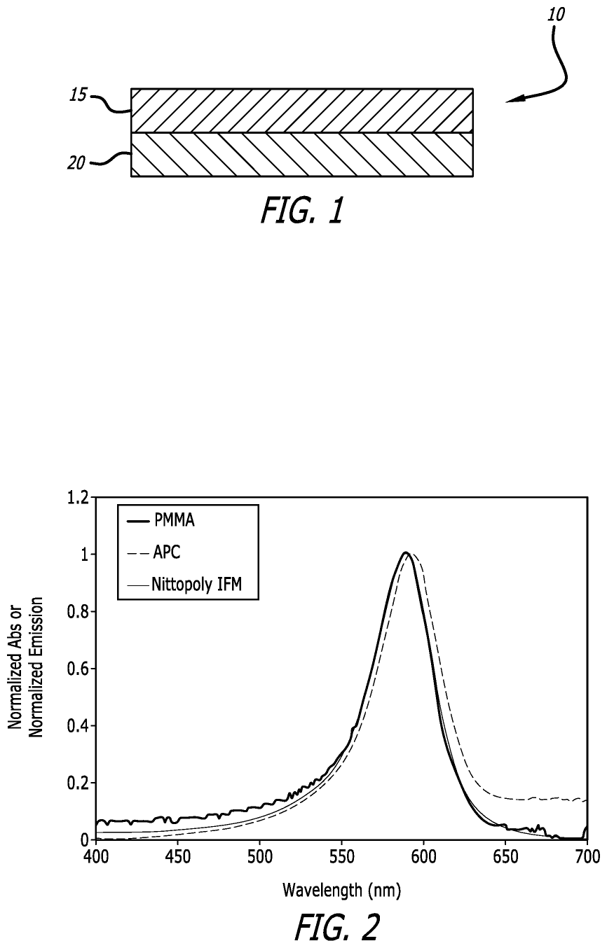

An example of a configuration of the device comprising a compound described herein is shown in FIG. 1. The device 10 can comprise the following layers in the order given: a filter layer 15 and a display layer 20. In some embodiments, the display layer can be the outermost layer or surface of a display device, e.g., an RGB source. Suitable RGB sources can be a liquid crystal display device, a plasma display panel and/or a cathode ray terminal. In some embodiments, the filter layer 15 can be positioned so that the RGB source is viewed through filter layer 15, e.g., on the distal or external side of the RGB source. In some embodiments, viewing the RGB source through the filter layer can increase the color distinction between the red and green colors.

The following embodiments are specifically contemplated herein:
Embodiment 1. A squarylium compound represented by a formula:

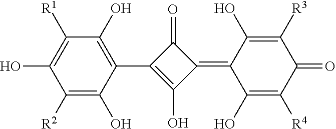

or a tautomer thereof; wherein IV, R², R³, and R⁴ are independently H, L, —CO-L, Ar, or -L-Ar, wherein each L is independently an acyclic C₁₋₆ hydrocarbon group, and each Ar is independently an optionally substituted C₆₋₁₀ aryl group.
Embodiment 2. The squarylium compound of embodiment 1, where all substituents of each Ar, if present, are represented by an empirical formula C₁₋₁₀H₃₋₂₁O_{0-1.}
Embodiment 3. The squarylium compound of embodiment 2, wherein R¹ is H.
Embodiment 4. The squarylium compound of embodiment 2, wherein R¹ is —COCH₃.
Embodiment 5. The squarylium compound of embodiment 2, wherein R¹ is linear C₁₋₄ alkyl.
Embodiment 6. The squarylium compound of embodiment 2, wherein R¹ is linear C₃₋₄ alkenyl.
Embodiment 7. The squarylium compound of embodiment 2, wherein R¹ is optionally substituted —CH₂-phenyl.
Embodiment 8. The squarylium compound of embodiment 2, wherein R¹ is optionally substituted phenyl.
Embodiment 9. The squarylium compound of embodiment 2, wherein R¹ is optionally substituted —CH₂CH═CH-phenyl.

Embodiment 10. The squarylium compound of embodiment 1, 2, 3, 4, 5, 6, 7, 8, or 9, which is not:

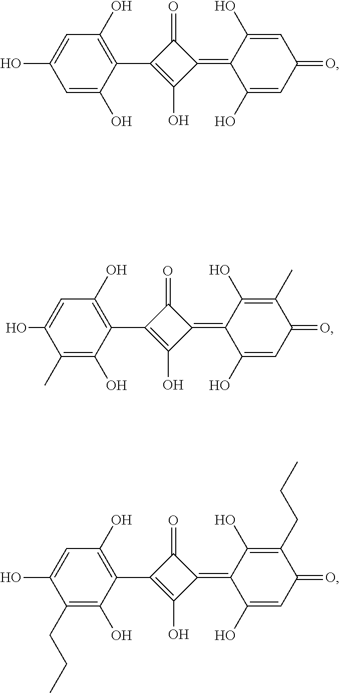 [SMILES: O=C1C=C(O)C(=C2C(=O)C(c3c(O)cc(O)cc3O)=C2O)C(O)=C1 ; CC1=C(O)/C(=C2\C(=O)C(c3c(O)cc(O)c(C)c3O)=C2O)C(O)=CC1=O ; CCCC1=C(O)/C(=C2\C(=O)C(c3c(O)cc(O)c(CCC)c3O)=C2O)C(O)=CC1=O]

or a tautomer thereof.
Embodiment 11. The squarylium compound of embodiment, 1, 2, 3, 4, 5, 6, 7, 8, 9, or 10, wherein R² is —CO-L, Ar, or -L-Ar.
Embodiment 12. The squarylium compound of embodiment 1, 2, 3, 4, 5, 6, 7, 8, 9, 10, wherein R² is H, and R⁴ is L, —CO-L, Ar, or -L-Ar.
Embodiment 13. The squarylium compound of embodiment 1, 2, 3, 4, 5, 6, 7, 8, 9, 10, wherein R² is CH₂CH₃, acyclic C₄₋₆ alkyl, or an acyclic C₁₋₆ hydrocarbyl that is not alkyl, and R⁴ is H, L, —CO-L, Ar, or -L-Ar.
Embodiment 14. The squarylium compound of embodiment 1, 2, 3, 4, 5, 6, 7, 8, 9, 10, wherein R² is CH₃ and R⁴ is H, an acyclic C₂₋₆ hydrocarbyl , —CO-L, Ar, or -L-Ar.
Embodiment 15. The squarylium compound of embodiment 1, 2, 3, 4, 5, 6, 7, 8, 9, 10, wherein R² is C₃ alkyl and R⁴ is H, C₁₋₂ alkyl, an acyclic C₄₋₆ alkyl, an acyclic C₂₋₆ hydrocarbyl that is not alkyl, —CO-L, Ar, or -L-Ar.
Embodiment 16. The squarylium compound of embodiment 1, 2, 3, 4, 5, 6, 7, 8, 9, 10, wherein R² and R⁴ are independently an acyclic C₄₋₆ alkyl, an acyclic C₂₋₆ hydrocarbyl that is not alkyl, —CO-L, Ar, or -L-Ar.
Embodiment 17. The squarylium compound of embodiment 1, that is:

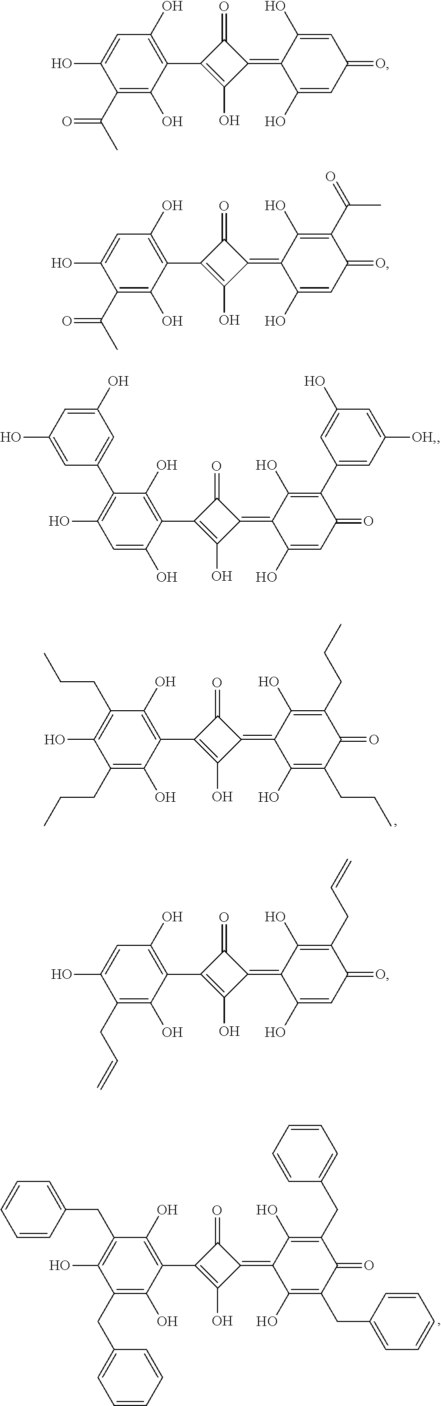

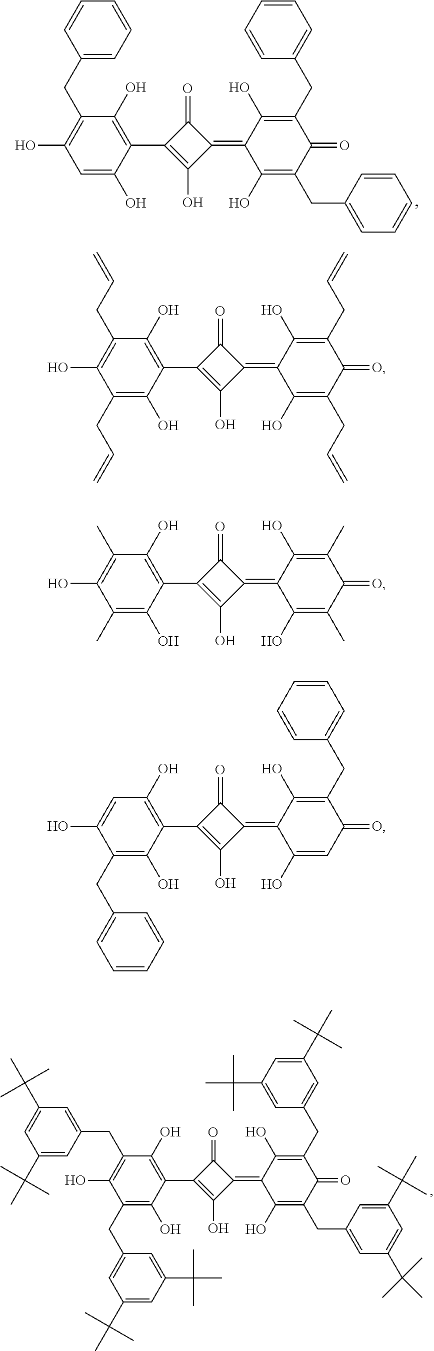

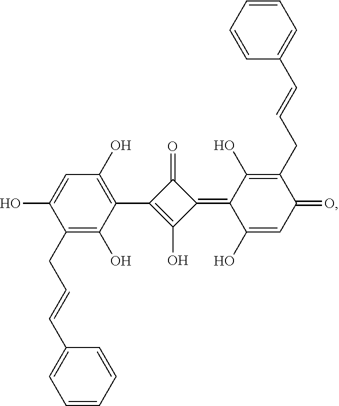 [SMILES: O=C1C=C(O)/C(=C2/C(=O)C(c3c(O)cc(O)c(C/C=C/c4ccccc4)c3O)=C2O)C(O)=C1C/C=C/c1ccccc1]

or a tautomer thereof.
Embodiment 18. The squarylium compound of embodiment 1, that is:

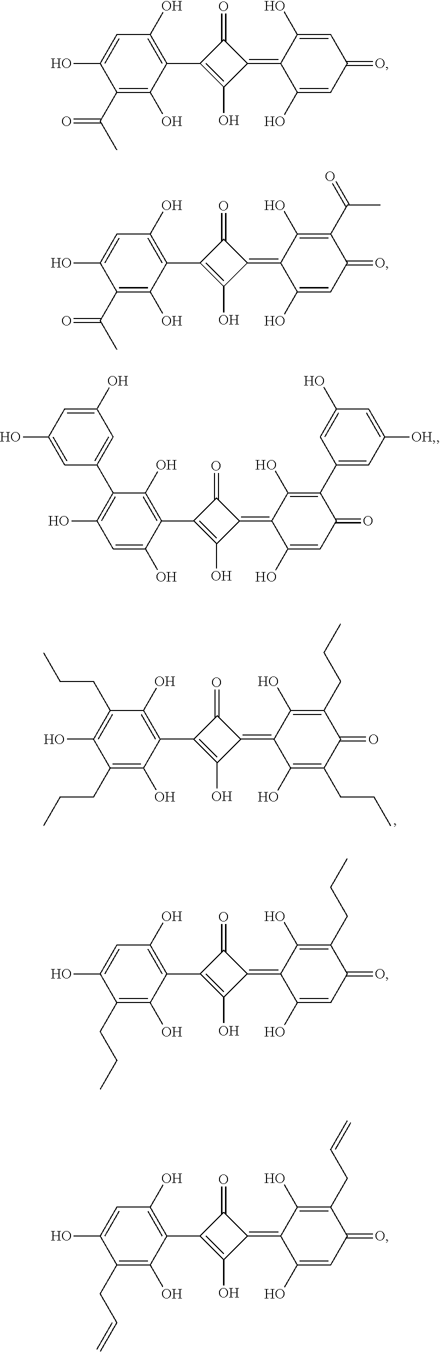

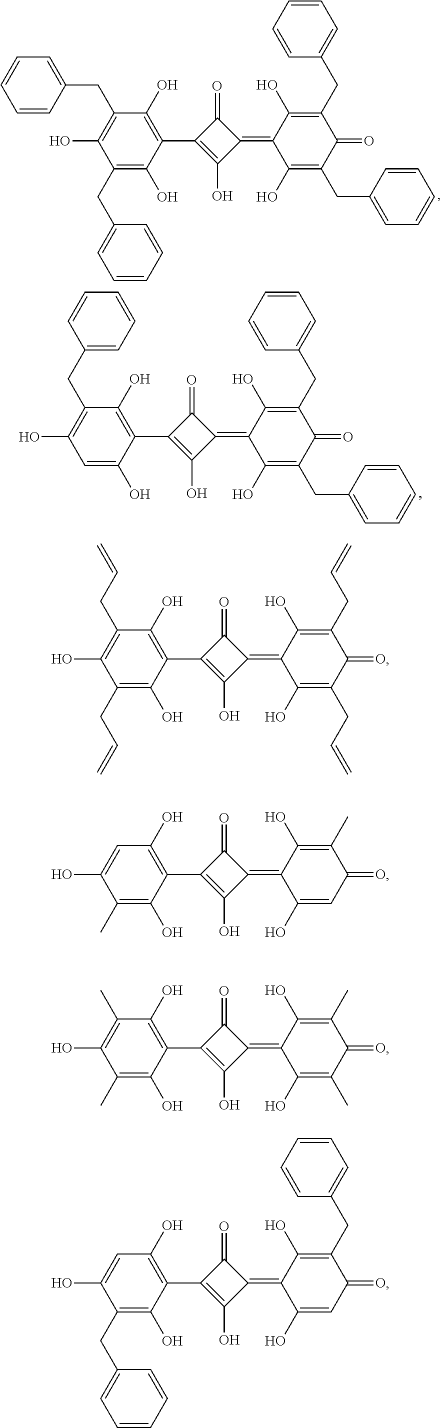

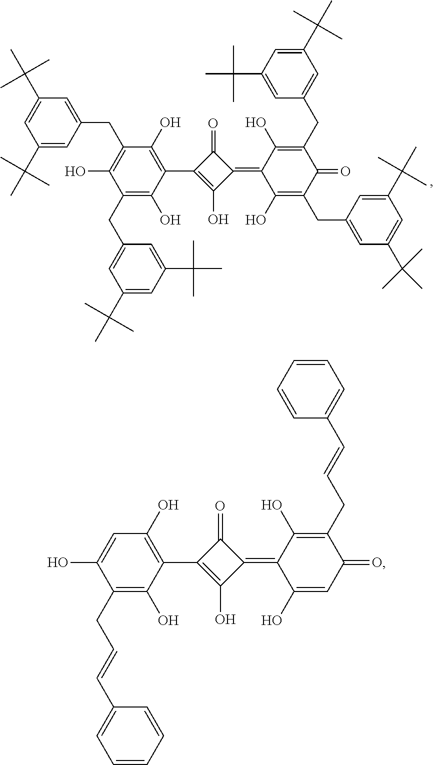 [SMILES: CC(C)(C)c1cc(CC2=C(O)C(=C3C(=O)C(c4c(O)c(Cc5cc(C(C)(C)C)cc(C(C)(C)C)c5)c(O)c(Cc5cc(C(C)(C)C)cc(C(C)(C)C)c5)c4O)=C3O)C(O)=C(Cc3cc(C(C)(C)C)cc(C(C)(C)C)c3)C2=O)cc(C(C)(C)C)c1 ; O=C1C=C(O)/C(=C2/C(=O)C(c3c(O)cc(O)c(C/C=C/c4ccccc4)c3O)=C2O)C(O)=C1C/C=C/c1ccccc1]

or a tautomer thereof.
Embodiment 19. An optical filter comprising:
   the squarylium compound of claim 1, 2, 3, 4, 5, 6, 7, 8, 9, 10, 11, 12, 13, 14, 15, 16, 17, or 18, and
   a polymer matrix, wherein the squarylium compound is disposed within the polymer matrix;
   wherein the filter has a quantum yield of less than about 1%.
Embodiment 20. The optical filter of embodiment 19, wherein the polymer matrix comprises poly(methyl methacrylate) (PMMA).
Embodiment 21. The optical filter of embodiment 19 or 20, wherein the polymer matrix comprises oxygen scavenging agent.
Embodiment 22. The optical filter of embodiment 19, 20, or 21, wherein the filter has a peak absorption of greater than 550 nm.
Embodiment 23. The optical filter of embodiment 22, wherein the filter has a peak absorbance wavelength of greater than 568 nm.
Embodiment 24. The optical filter of embodiment 19, 20, 21, 22, or 23, wherein the filter has a full width at half maximum (FWHM) of less than 50 nm.
Embodiment 25. The optical filter of embodiment 24, wherein the filter has a full width at half maximum (FWHM) of about 40 to about 50 nm.
Embodiment 26. A display device comprising the optical filter of embodiment 19, 20, 21, 22, 23, 24, or 25, and an RBG source positioned to allow viewing of the RGB source through the optical filter.

### EXAMPLES

The following are examples of some methods that may be used to prepare and use the compounds described herein.

### Example 1

### Synthesizing Squarylium Materials

### 8-1. Example of Synthesis

### Example 1.1

### Scheme 1.1 Synthesis of Squarylium Compound 7

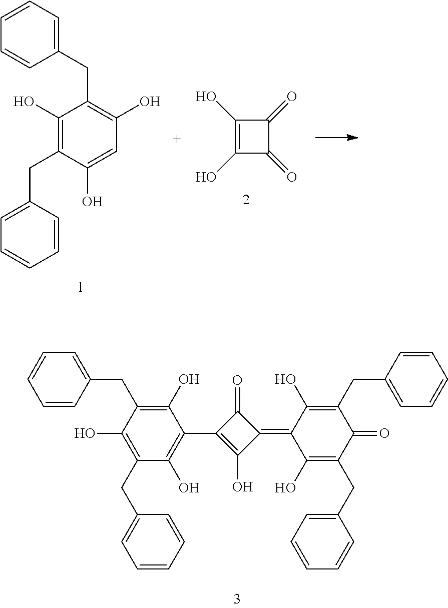 [SMILES: Oc1cc(O)c(Cc2ccccc2)c(O)c1Cc1ccccc1 ; O=c1c(O)c(O)c1=O ; O=C1C(Cc2ccccc2)=C(O)C(=C2C(=O)C(c3c(O)c(Cc4ccccc4)c(O)c(Cc4ccccc4)c3O)=C2O)C(O)=C1Cc1ccccc1]

Synthesis of Squarylium Compound 3: Phloroglucinol derivative 1 was prepared as described in Gisso, Arnaud, et al. Tetrahedron 60(32) 6807-6812 (2004). Phloroglucinol derivative 1 (1.70 g, 5.55 mmol) and squaric acid 2 (0.32 g, 2.81 mmol) were combined in acetic acid (50 mL), stirred, and heated to reflux for 24 hours. The mixture was cooled to room temperature, filtered, and washed with acetic acid (5 mL). The filter cake was dried at 70° C. in a vacuum oven to give 686 mg of squarylium compound 3(HPLC-MS[APCI-negative mode; samples prepared in MeOH with triethylamine included] m/z=690; ¹H NMR (DMSO-d₆, 400 MHz) δ− 3.90(s, 8H), 7.10-7.40 (m, 20H), 10.82 (s, 5H).

### Example 1.2

### Scheme 1.2 Synthesis of Squarylium Compounds 1-6, and 8-15

Synthesis of Squarylium Compounds 1-6, and 8-15: Squarylium compounds 1-6 and 8-15 were synthesized in a manner similar to that described with respect to squarylium compound 3 above, except that 2 equivalents of the precursor identified in Table 1 below was used in the place of Phloroglucinol derivative 1, or 1 equivalent each of the precursors with respect to squarylium compounds 1 and 8.

TABLE 1

| Squarylium | | | |
|---|---|---|---|
| Compound | Structure | Precursor | Citation |
| 1 | 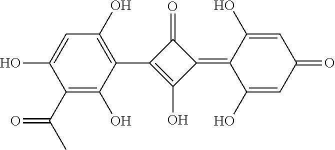 [SMILES: CC(=O)c1c(O)cc(O)c(C2=C(O)C(=C3C(O)=CC(=O)C=C3O)C2=O)c1O] | 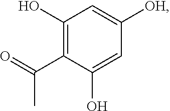 | Sigma Aldrich |
| | | 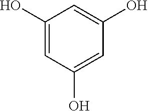 | Triebs, A., et al Chem. Int. Ed. Eng.; 1965, 4, 694 |
| 2 | 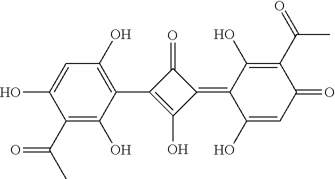 [SMILES: CC(=O)C1=C(O)/C(=C2\C(=O)C(c3c(O)cc(O)c(C(C)=O)c3O)=C2O)C(O)=CC1=O] | 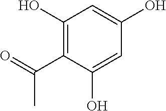 | Sigma Aldrich |
| 3 | 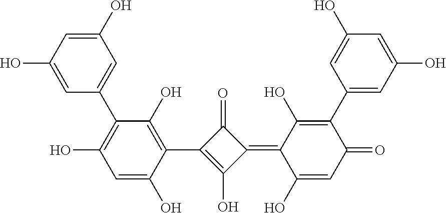 [SMILES: O=C1C=C(O)/C(=C2/C(=O)C(c3c(O)cc(O)c(-c4cc(O)cc(O)c4)c3O)=C2O)C(O)=C1c1cc(O)cc(O)c1] | 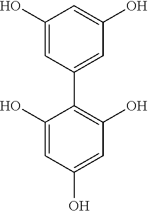 [SMILES: Oc1cc(O)cc(-c2c(O)cc(O)cc2O)c1] | Alfa-Aesar |
| 4 | 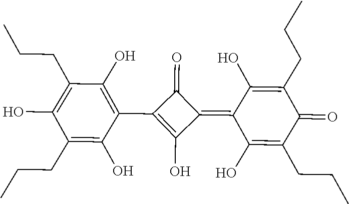 [SMILES: CCCC1=C(O)C(=C2C(=O)C(c3c(O)c(CCC)c(O)c(CCC)c3O)=C2O)C(O)=C(CCC)C1=O] | 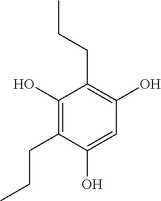 | Synthesized from 2,4- diallylphloro- glucinol, described below |
| 5 | 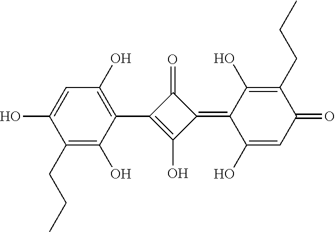 [SMILES: CCCC1=C(O)/C(=C2\C(=O)C(c3c(O)cc(O)c(CCC)c3O)=C2O)C(O)=CC1=O] | 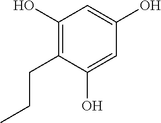 | Synthesized from 2,4- diallylphloro- glucinol, described below |
| 6 | 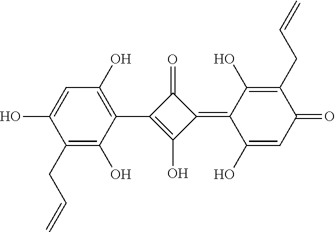 [SMILES: C=CCC1=C(O)/C(=C2\C(=O)C(c3c(O)cc(O)c(CC=C)c3O)=C2O)C(O)=CC1=O] | 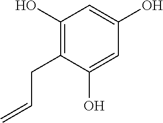 | Gisso, Arnaud, et al, Tetrahedron, 60(32) 6807- 6812 (2004) |
| 7 | 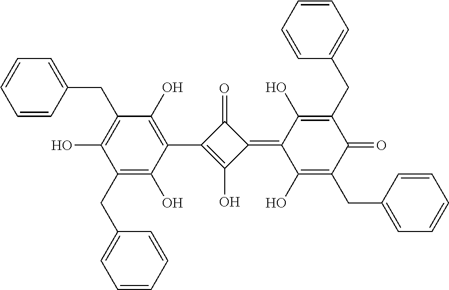 [SMILES: O=C1C(Cc2ccccc2)=C(O)C(=C2C(=O)C(c3c(O)c(Cc4ccccc4)c(O)c(Cc4ccccc4)c3O)=C2O)C(O)=C1Cc1ccccc1] | 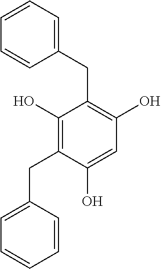 [SMILES: Oc1cc(O)c(Cc2ccccc2)c(O)c1Cc1ccccc1] | Gisso, Arnaud, et al, Tetrahedron, 60(32) 6807- 6812 (2004) |
| 8 | 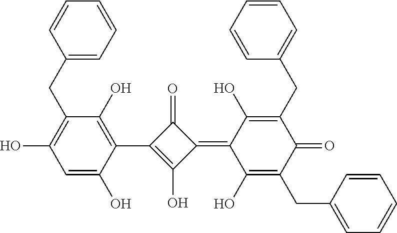 [SMILES: O=C1C(Cc2ccccc2)=C(O)C(=C2C(=O)C(c3c(O)cc(O)c(Cc4ccccc4)c3O)=C2O)C(O)=C1Cc1ccccc1] | 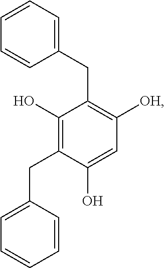 [SMILES: Oc1cc(O)c(Cc2ccccc2)c(O)c1Cc1ccccc1] | Gisso, Arnaud, et al, Tetrahedron, 60(32) 6807- 6812 (2004) |
| | | 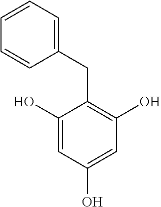 [SMILES: Oc1cc(O)c(Cc2ccccc2)c(O)c1] | Gisso, Arnaud, et al, Tetrahedron, 60(32) 6807- 6812 (2004) |
| 9 | 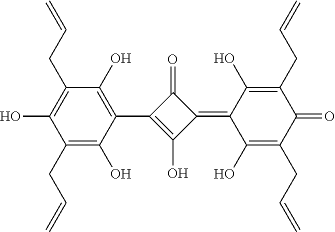 [SMILES: C=CCC1=C(O)C(=C2C(=O)C(c3c(O)c(CC=C)c(O)c(CC=C)c3O)=C2O)C(O)=C(CC=C)C1=O] | 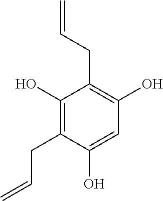 | Gisso, Arnaud, et al, Tetrahedron, 60(32) 6807- 6812 (2004) |
| 10 | 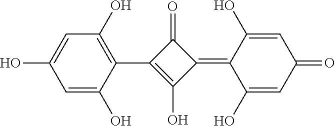 [SMILES: O=C1C=C(O)C(=C2C(=O)C(c3c(O)cc(O)cc3O)=C2O)C(O)=C1] | 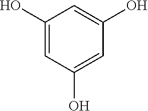 | Triebs, A., et al Chem. Int. Ed. Eng.; 1965, 4, 694 |
| 11 | 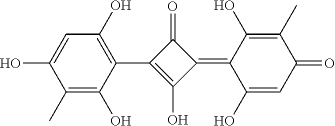 [SMILES: CC1=C(O)/C(=C2\C(=O)C(c3c(O)cc(O)c(C)c3O)=C2O)C(O)=CC1=O] | 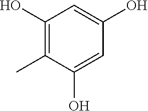 | Sigma Aldrich |
| 12 | 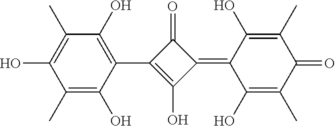 [SMILES: CC1=C(O)C(=C2C(=O)C(c3c(O)c(C)c(O)c(C)c3O)=C2O)C(O)=C(C)C1=O] | 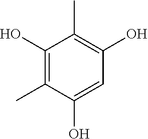 | Dittmer, C., et al, Eur. J. Org. Chem., 2007, 35, 5886-5898 |
| 13 | 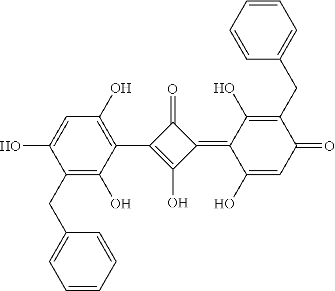 [SMILES: O=C1C=C(O)/C(=C2/C(=O)C(c3c(O)cc(O)c(Cc4ccccc4)c3O)=C2O)C(O)=C1Cc1ccccc1] | 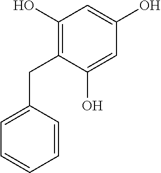 [SMILES: Oc1cc(O)c(Cc2ccccc2)c(O)c1] | Triebs, A., et al Chem. Int. Ed. Eng.; 1965, 4, 694 |
| 14 | 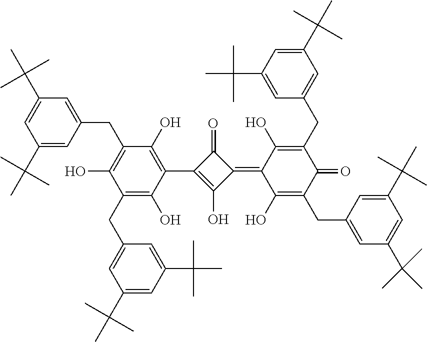 [SMILES: CC(C)(C)c1cc(CC2=C(O)C(=C3C(=O)C(c4c(O)c(Cc5cc(C(C)(C)C)cc(C(C)(C)C)c5)c(O)c(Cc5cc(C(C)(C)C)cc(C(C)(C)C)c5)c4O)=C3O)C(O)=C(Cc3cc(C(C)(C)C)cc(C(C)(C)C)c3)C2=O)cc(C(C)(C)C)c1] | 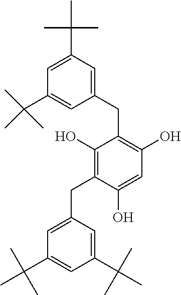 [SMILES: CC(C)(C)c1cc(Cc2c(O)cc(O)c(Cc3cc(C(C)(C)C)cc(C(C)(C)C)c3)c2O)cc(C(C)(C)C)c1] | See below |
| 15 | 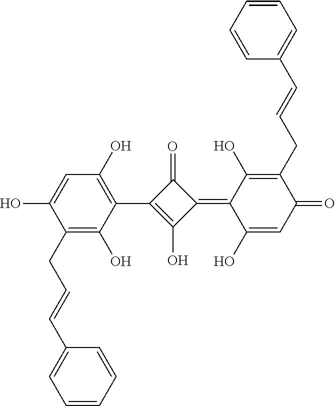 [SMILES: O=C1C=C(O)/C(=C2/C(=O)C(c3c(O)cc(O)c(C/C=C/c4ccccc4)c3O)=C2O)C(O)=C1C/C=C/c1ccccc1] | 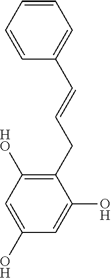 [SMILES: Oc1cc(O)c(C/C=C/c2ccccc2)c(O)c1] | Gisso, Arnaud, et al, Tetrahedron, 60(32) 6807- 6812 (2004) |

### Example 1.3

### Scheme 1.3 Synthesis of Precursors for Squarylium Compounds 4 and 5

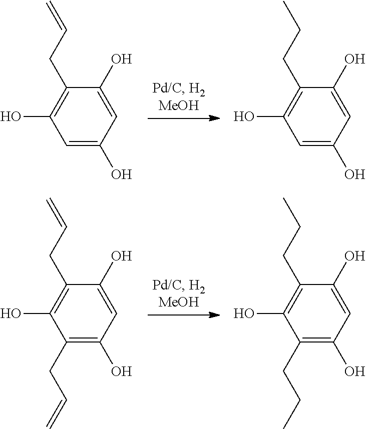

Synthesis of Precursors for Squarylium Compounds 4 and 5: Either 2-allylphloroglucinol or 2, 4-diallylphloroglucinol was dissolved in methanol (10 mL/g). The afforded solution was degassed, establishing an argon atmosphere. 10% palladium-on-carbon (water-wet grade) was added (0.3 g catalyst/g) and a hydrogen atmosphere was established at ambient pressure. The reaction was stirred for 2 hours to completion. The hydrogen atmosphere was exchanged for an argon atmosphere. The catalyst was then removed by filtration and the filtrate was concentrated. The desired product was confirmed by LC-MS (APCI): mz 167 or 209.

### Example 1.4

### Scheme 1.4 Synthesis of Precursors for Squarylium Compound 14

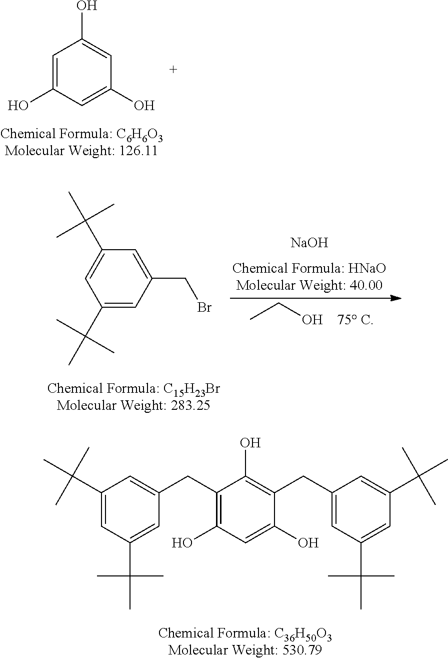

Synthesis of Precursors for Squarylium Compound 14:

41-(bromomethyl)-3,5-di-tert-butylbenzene (4.94 g, 17.4 mmol) and phlorogucinol (1.10 g, 8.7 mmol) was added to a stirring quantity of 25 mL of ethanol at room temperature. The mixture was stirred until complete dissolution of ingredients. Subsequently, sodium hydroxide (0.697 g, 17.4 mmol) was added. The reaction was heated at 75° C. for six hours. The reaction was checked by TLC (9Hex: 1 Eth Aoc), which indicated completion of reaction. The contents of the flask were filtered and extracted with 300 mL of water and 300 mL of dichloromethane. The organic layer was rotovaped. No further purification was carried out. 2 g of an orange powder was produced (43% yield).

### Example 1.5

Scheme 1.5 Synthesis of Squarylium Compound 8 from Squarylium Compound 13

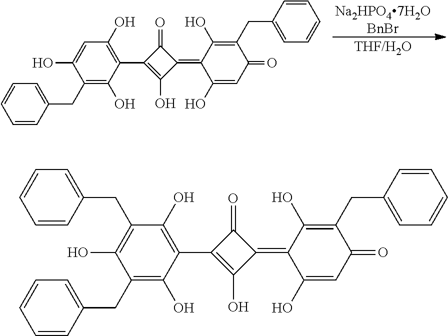 [SMILES: O=C1C=C(O)/C(=C2/C(=O)C(c3c(O)cc(O)c(Cc4ccccc4)c3O)=C2O)C(O)=C1Cc1ccccc1 ; "Na2HPO4·7H2OBnBr" ; "THF/H2O" ; O=C1C=C(O)/C(=C2/C(=O)C(c3c(O)c(Cc4ccccc4)c(O)c(Cc4ccccc4)c3O)=C2O)C(O)=C1Cc1ccccc1]

Synthesis of Squarylium Compound 8 from Squarylium Compound 13:

Squarylium compound 13 (232 mg), disodium phosphate heptahydrate (750 mg), water (5 mL), and tetrahydrofuran (5 mL) were combined and stirred under argon. Benzyl bromide was added (95 mg) and the mixture was heated at 60° C. for eighteen hours. Another 110 mg benzyl bromide was added and the reaction continued for 6 additional hours. The reaction mixture was extracted with ethyl acetate. The extract was washed with brine, dried with sodium sulfate, and concentrated. The concentrate was loaded onto a 40-g silica gel column and a methanol-dichloromethane eluent was applied, linearly increasing the percentage of methanol to 10% over 15 column volumes. Doing so produced several fractions of the mixture that contained pure material. These were concentrated to 19 mg of pure tri-benzylated product. The desired product was confirmed by LC-MS (APCI): mz 600.

### Example 1.6

### Scheme 1.6 Synthesis of Squarylium Compound 14

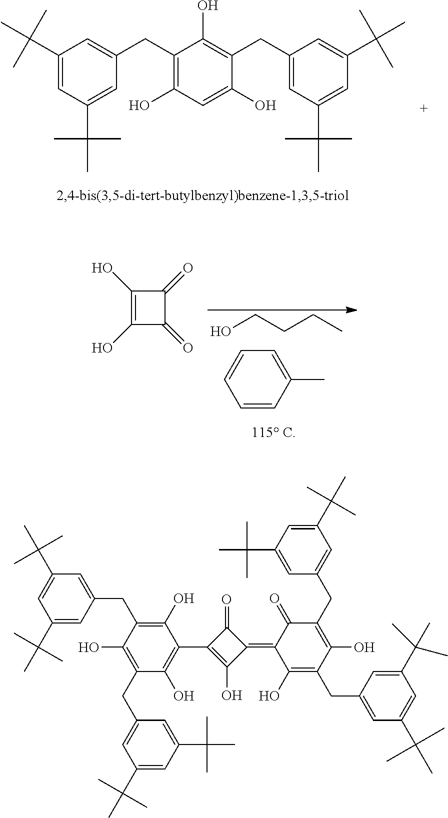

### Synthesis of Squarylium Compound 14:

2,4-bis(3,5-di-tert-butylbenzyl)benzene-1.3.5-triol (Squarylium Compound 14 precursor) (0.280 g, 0.53 mmol) and squaric acid (0.030 g, 0.26 mmol) was added to a stirring quantity of 10 mL of n-butanol and toluene (1:1 v/v ratio). A small scoop of 4A, 8-12 mesh molecular sieves (about 280 mg) was then added. The flask was equipped with a reflux condenser and the set-up subjected to a 115° C., pre-heated bath. After about 38 hours of reaction, the reaction mixture was extracted with 40 mL of water and 40 mL ether. The organic layer was collected and rotovaped. No further purification was carried out. 50 mg of a deep navy colored material was produced. Yield was 17%.

### Example 2.1

### Fabrication of Filter Layer

A glass substrate was prepared in substantially the following manner. A 1.1 mm thick glass substrate measuring 1 inch×1 inch was cut to size. The glass substrate was then washed with detergent and deionized (DI) water, rinsed with fresh DI water, and sonicated for about 1 hour. The glass was then soaked in isopropanol (IPA) and sonicated for about 1 hour. The glass substrate was then soaked in acetone and sonicated for about 1 hour. The glass was then removed from the acetone bath and dried with nitrogen gas at room temperature.

A 25 wt % solution of Poly(methyl methacrylate) (PMMA) (average M.W. 120,000 by GPC from Sigma Aldrich) copolymer in cyclopentanone (99.9% pure) was prepared. The prepared copolymer was stirred overnight at 40° C. [PMMA] CAS: 9011-14-7; [Cyclopentanone] CAS: 120-92-3

The 25% PMMA solution prepared above (4 g) was added to 3 mg of squarylium compound 1 made as described above in a sealed container, and mixed for about 30 minutes. The PMMA/Chromophore solution was then spin coated onto a prepared glass substrate at 1000 RPM for 3 s; then 1500 RPM for 20 s and then 500 RPM for 2 s. The resulting wet coating had a thickness of about 10 um. The samples were covered with aluminum foil before spin coating to protect them from exposure to light. Three samples each were prepared in this manner for each quantum yield and/or stability study. The spin coated samples were baked in a vacuum oven at 80° C. for 3 hours to evaporate the remaining solvent.

The 1 inch×1 inch sample was inserted into a Shimadzu, UV-3600 UV-VIS-NIR spectrophotometer (Shimadzu Instruments, Inc., Columbia, Md., USA). All device operation was performed inside a nitrogen-filled glove-box. The resulting absorption spectrum is shown in FIG. 2. The maximum absorption was normalized at about 100% at a wavelength of 568 nm (the perceived maximum absorbance wavelength), and the half-value width (FWHM) at the maximum absorption was 56 nm.

The fluorescence spectrum of a 1 inch×1 inch film sample prepared as described above was determined using a Fluorolog spectrofluorometer (Horiba Scientific, Edison, N.J., USA) with the excitation wavelength set at the respective maximum absorbance wavelength.

The quantum yield of a 1 inch×1 inch sample prepared as described above were determined using a Quantarus-QY spectrophotometer (Hamamatsu Inc., Campbell, Calif., USA) set at the respective maximum absorbance wavelength. The quenching compounds of the invention were weakly fluorescent or essentially non-fluorescent.

The results of the film characterization (absorption peak wavelength, FWHM, and quantum yield) are shown in Table 2 below.

TABLE 2

| | | Peak | | |
|---|---|---|---|---|
| Squarylium | | absorption | | Quantum |
| Compound | Structure | (nm) | FWHM (nm) | yield |
| 1 | 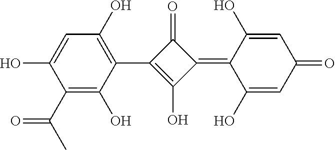 [SMILES: CC(=O)c1c(O)cc(O)c(C2=C(O)C(=C3C(O)=CC(=O)C=C3O)C2=O)c1O] | 568 nm | 56 | 0.4% |
| 2 | 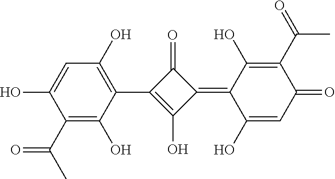 [SMILES: CC(=O)C1=C(O)/C(=C2\C(=O)C(c3c(O)cc(O)c(C(C)=O)c3O)=C2O)C(O)=CC1=O] | N/A | N/A | 0.5% |
| 3 | 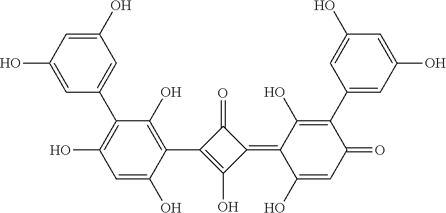 [SMILES: O=C1C=C(O)/C(=C2/C(=O)C(c3c(O)cc(O)c(-c4cc(O)cc(O)c4)c3O)=C2O)C(O)=C1c1cc(O)cc(O)c1] | 581 nm | 38 | 0.4% |
| 4 | 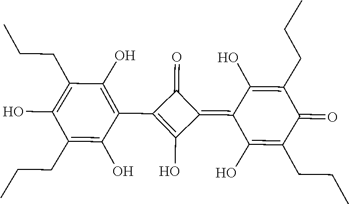 [SMILES: CCCC1=C(O)C(=C2C(=O)C(c3c(O)c(CCC)c(O)c(CCC)c3O)=C2O)C(O)=C(CCC)C1=O] | 579 nm | 46 | 0.4% |
| 5 | 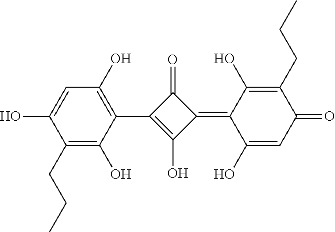 [SMILES: CCCC1=C(O)/C(=C2\C(=O)C(c3c(O)cc(O)c(CCC)c3O)=C2O)C(O)=CC1=O] | 579 nm | 46 | 0.3% |
| 6 | 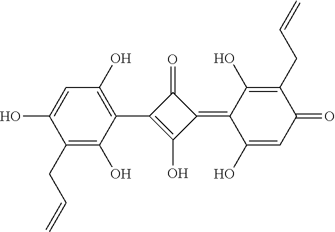 [SMILES: C=CCC1=C(O)/C(=C2\C(=O)C(c3c(O)cc(O)c(CC=C)c3O)=C2O)C(O)=CC1=O] | 578 nm | 54 | 0.4% |
| 7 | 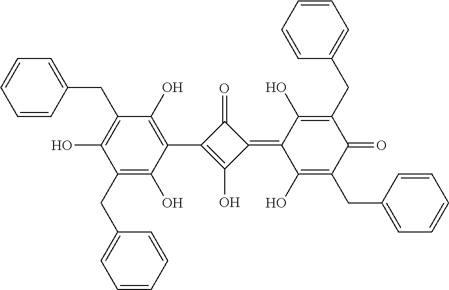 [SMILES: O=C1C(Cc2ccccc2)=C(O)C(=C2C(=O)C(c3c(O)c(Cc4ccccc4)c(O)c(Cc4ccccc4)c3O)=C2O)C(O)=C1Cc1ccccc1] | 588 nm | 42 | 0.4% |
| 8 | 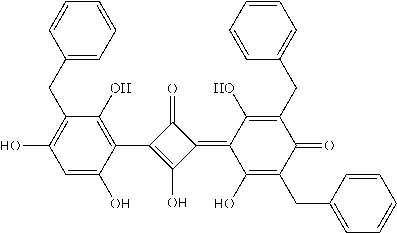 [SMILES: O=C1C(Cc2ccccc2)=C(O)C(=C2C(=O)C(c3c(O)cc(O)c(Cc4ccccc4)c3O)=C2O)C(O)=C1Cc1ccccc1] | 584 nm | 44 | 0.4% |
| 9 | 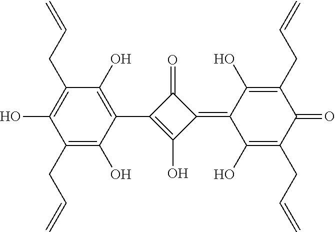 [SMILES: C=CCC1=C(O)C(=C2C(=O)C(c3c(O)c(CC=C)c(O)c(CC=C)c3O)=C2O)C(O)=C(CC=C)C1=O] | 582 nm | 44 | 0.3% |
| 10 | 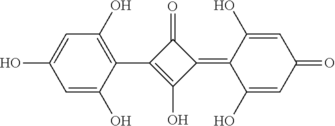 [SMILES: O=C1C=C(O)C(=C2C(=O)C(c3c(O)cc(O)cc3O)=C2O)C(O)=C1] | 575 nm | 48 nm | 0.4% |
| 11 | 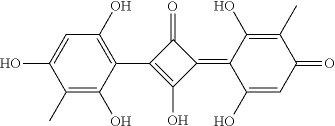 [SMILES: CC1=C(O)/C(=C2\C(=O)C(c3c(O)cc(O)c(C)c3O)=C2O)C(O)=CC1=O] | 578 nm | 45 nm | 0.6% |
| 12 | 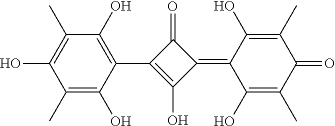 [SMILES: CC1=C(O)C(=C2C(=O)C(c3c(O)c(C)c(O)c(C)c3O)=C2O)C(O)=C(C)C1=O] | 583 nm | 57 nm | 0.4% |
| 13 | 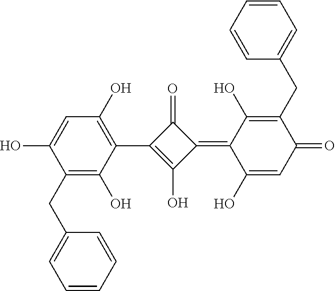 [SMILES: O=C1C=C(O)/C(=C2/C(=O)C(c3c(O)cc(O)c(Cc4ccccc4)c3O)=C2O)C(O)=C1Cc1ccccc1] | 580 nm | 42 nm | 0.4% |
| 14 | 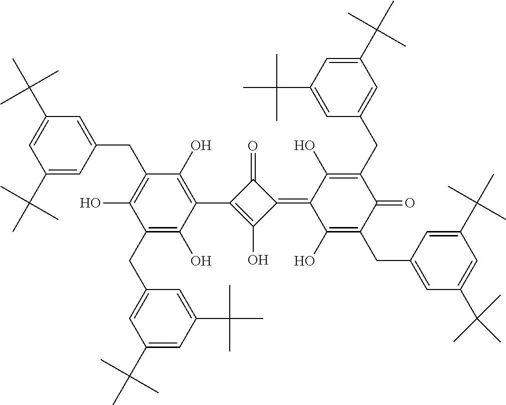 [SMILES: CC(C)(C)c1cc(CC2=C(O)C(=C3C(=O)C(c4c(O)c(Cc5cc(C(C)(C)C)cc(C(C)(C)C)c5)c(O)c(Cc5cc(C(C)(C)C)cc(C(C)(C)C)c5)c4O)=C3O)C(O)=C(Cc3cc(C(C)(C)C)cc(C(C)(C)C)c3)C2=O)cc(C(C)(C)C)c1] | | | |
| 15 | 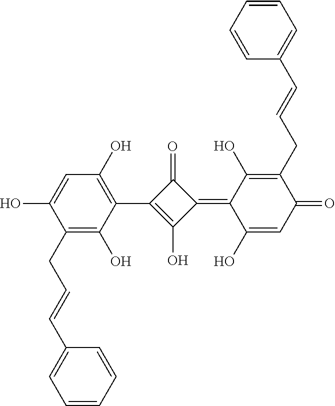 [SMILES: O=C1C=C(O)/C(=C2/C(=O)C(c3c(O)cc(O)c(C/C=C/c4ccccc4)c3O)=C2O)C(O)=C1C/C=C/c1ccccc1] | | | |

Thus at least Squarylium Compounds 1 and 3-13 demonstrated their effectiveness as a filter material useful in display devices.

It will be understood by those of skill in the art that numerous and various modifications can be made without departing from the spirit of the present invention. Therefore, it should be clearly understood that the forms of the present invention are illustrative only and are not intended to limit the scope of the present invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein.

Accordingly, the claims are not limited to embodiments precisely as shown and described.

1. A squarylium compound represented by a formula: 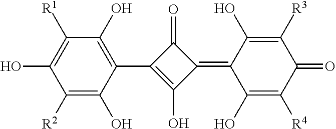
or a tautomer thereof;
wherein R¹, R², and R³ are independently H, L, —CO-L, Ar, or -L-Ar;
and R⁴ is independently L, —CO-L, Ar, or -L-Ar;
wherein each L is independently an acyclic C₁₋₆ hydrocarbon group, and each Ar is independently an optionally substituted C₆₋₁₀ aryl group;
wherein the squarylium compound is not 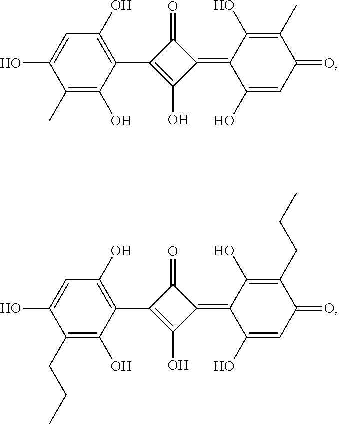 [SMILES: CC1=C(O)/C(=C2\C(=O)C(c3c(O)cc(O)c(C)c3O)=C2O)C(O)=CC1=O ; CCCC1=C(O)/C(=C2\C(=O)C(c3c(O)cc(O)c(CCC)c3O)=C2O)C(O)=CC1=O]
or a tautomer thereof.

2. The squarylium compound of claim 1, where each substituent of each Ar, if present, are represented by an empirical formula OH, or C₁₋₁₀H₃₋₂₁O₀₋₁.

3. The squarylium compound of claim 2, wherein R¹ is H.

4. The squarylium compound of claim 2, wherein R¹ is —COCH₃.

5. The squarylium compound of claim 2, wherein R¹ is linear C₁₋₄ alkyl.

6. The squarylium compound of claim 2, wherein R¹ is linear C₃₋₄ alkenyl.

7. The squarylium compound of claim 2, wherein R¹ is optionally substituted —CH₂-phenyl.

8. The squarylium compound of claim 2, wherein R¹ is optionally substituted phenyl.

9. The squarylium compound of claim 2, wherein R¹ is optionally substituted —CH₂CH═CH-phenyl.

10. The squarylium compound of claim 1, that is: 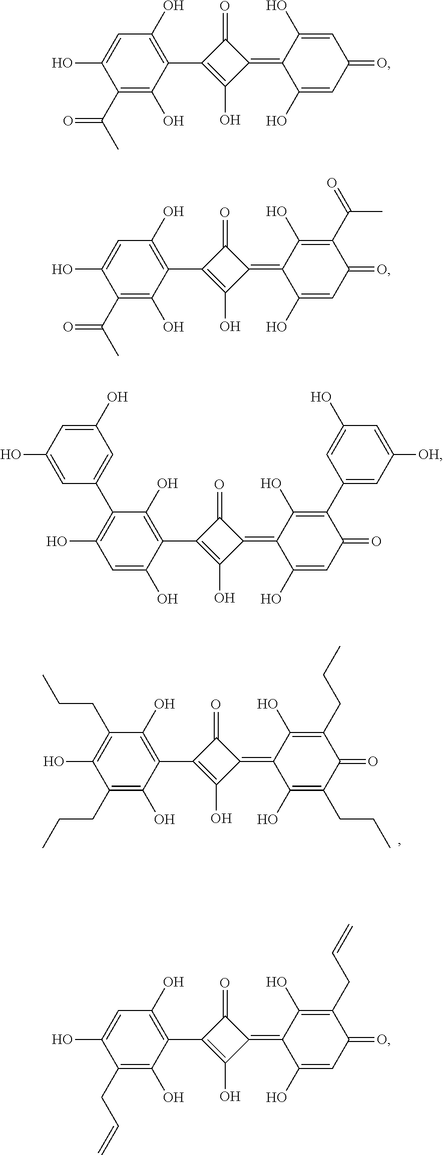 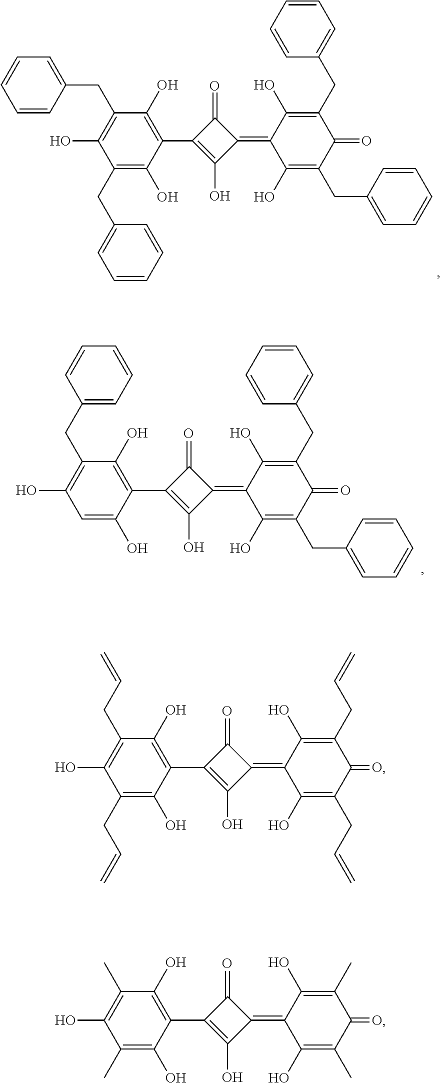 [SMILES: O=C1C(Cc2ccccc2)=C(O)C(=C2C(=O)C(c3c(O)c(Cc4ccccc4)c(O)c(Cc4ccccc4)c3O)=C2O)C(O)=C1Cc1ccccc1 ; O=C1C(Cc2ccccc2)=C(O)C(=C2C(=O)C(c3c(O)cc(O)c(Cc4ccccc4)c3O)=C2O)C(O)=C1Cc1ccccc1 ; C=CCC1=C(O)C(=C2C(=O)C(c3c(O)c(CC=C)c(O)c(CC=C)c3O)=C2O)C(O)=C(CC=C)C1=O ; CC1=C(O)C(=C2C(=O)C(c3c(O)c(C)c(O)c(C)c3O)=C2O)C(O)=C(C)C1=O] 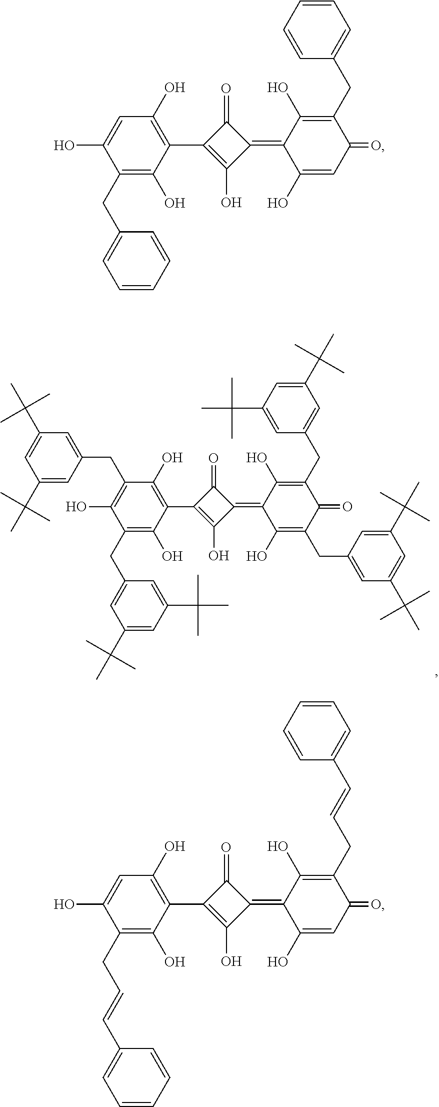 [SMILES: O=C1C=C(O)/C(=C2/C(=O)C(c3c(O)cc(O)c(Cc4ccccc4)c3O)=C2O)C(O)=C1Cc1ccccc1 ; CC(C)(C)c1cc(CC2=C(O)C(=C3C(=O)C(c4c(O)c(Cc5cc(C(C)(C)C)cc(C(C)(C)C)c5)c(O)c(Cc5cc(C(C)(C)C)cc(C(C)(C)C)c5)c4O)=C3O)C(O)=C(Cc3cc(C(C)(C)C)cc(C(C)(C)C)c3)C2=O)cc(C(C)(C)C)c1 ; O=C1C=C(O)/C(=C2/C(=O)C(c3c(O)cc(O)c(C/C=C/c4ccccc4)c3O)=C2O)C(O)=C1C/C=C/c1ccccc1]
or a tautomer thereof.

11. An optical filter comprising:
a squarylium compound, and
a polymer matrix, wherein the squarylium compound is disposed within the polymer matrix;
wherein the filter has a quantum yield of less than about 1%;
wherein the squarylium compound is represented by a formula: 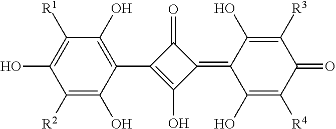
or a tautomer thereof;
wherein R¹, R², R³, and R⁴ are independently H, L, —CO-L, Ar, or -L-Ar, wherein each L is independently an acyclic C₁₋₆ hydrocarbon group, and each Ar is independently an optionally substituted C₆₋₁₀ aryl group.

12. The optical filter of claim 11, wherein the squarylium compound is: 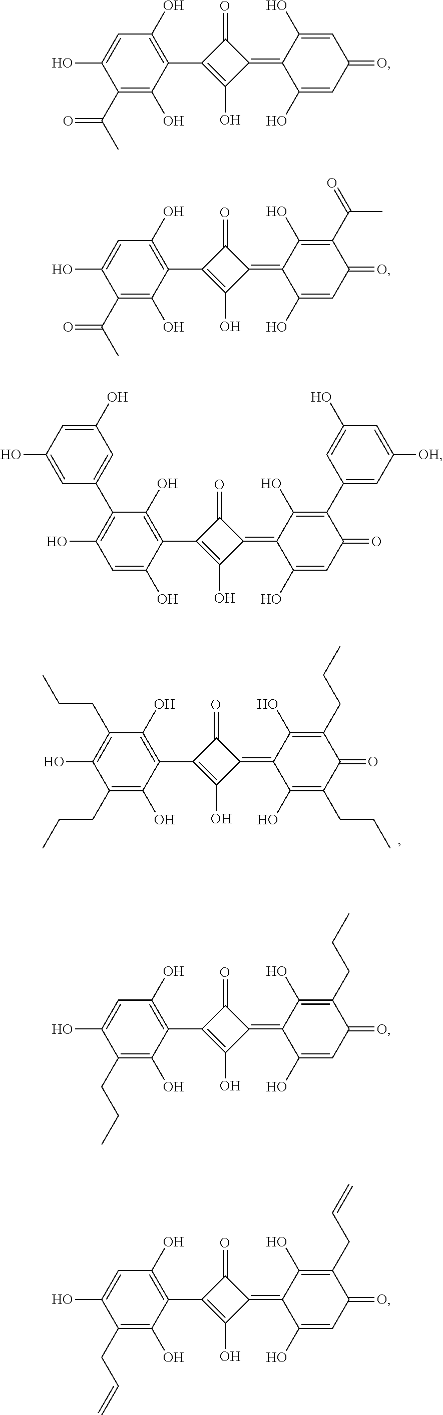 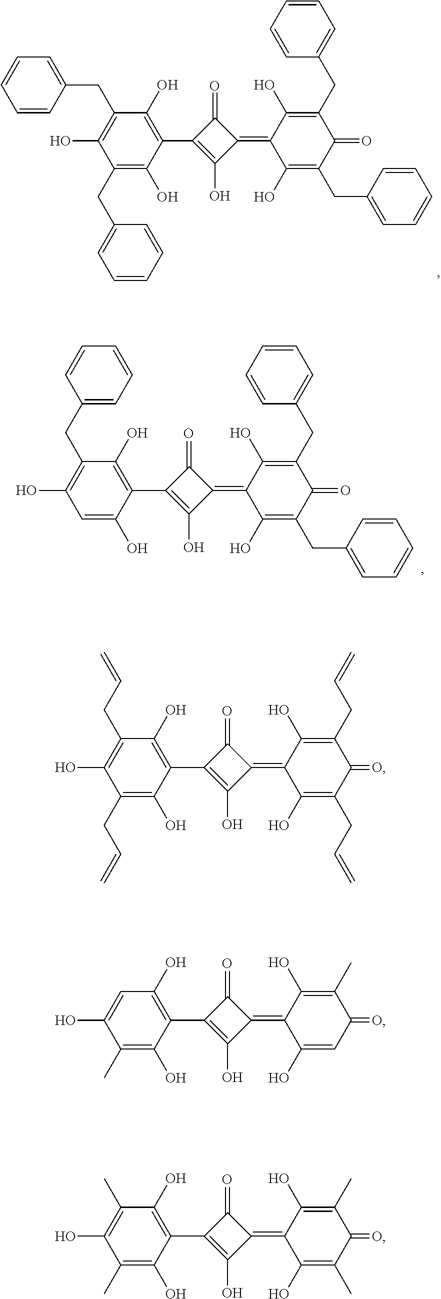 [SMILES: O=C1C(Cc2ccccc2)=C(O)C(=C2C(=O)C(c3c(O)c(Cc4ccccc4)c(O)c(Cc4ccccc4)c3O)=C2O)C(O)=C1Cc1ccccc1 ; O=C1C(Cc2ccccc2)=C(O)C(=C2C(=O)C(c3c(O)cc(O)c(Cc4ccccc4)c3O)=C2O)C(O)=C1Cc1ccccc1 ; C=CCC1=C(O)C(=C2C(=O)C(c3c(O)c(CC=C)c(O)c(CC=C)c3O)=C2O)C(O)=C(CC=C)C1=O ; CC1=C(O)/C(=C2\C(=O)C(c3c(O)cc(O)c(C)c3O)=C2O)C(O)=CC1=O ; CC1=C(O)C(=C2C(=O)C(c3c(O)c(C)c(O)c(C)c3O)=C2O)C(O)=C(C)C1=O] 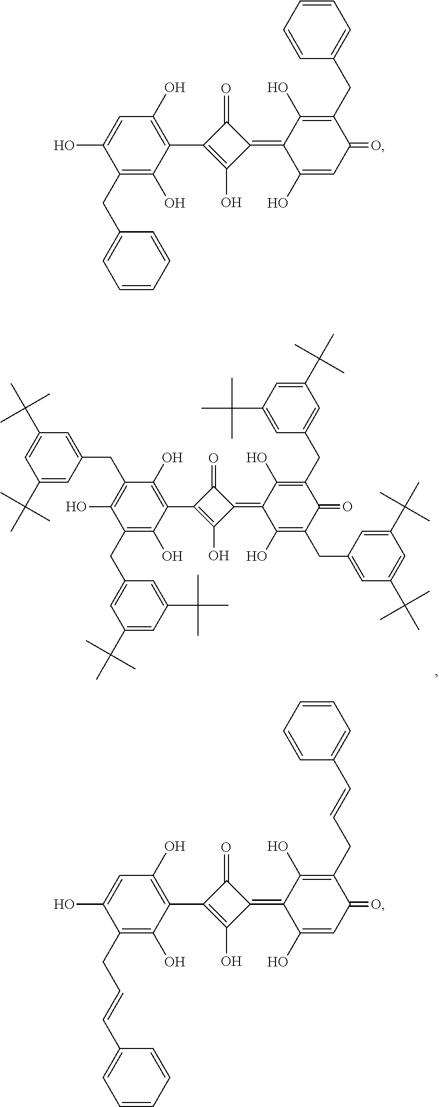 [SMILES: O=C1C=C(O)/C(=C2/C(=O)C(c3c(O)cc(O)c(Cc4ccccc4)c3O)=C2O)C(O)=C1Cc1ccccc1 ; CC(C)(C)c1cc(CC2=C(O)C(=C3C(=O)C(c4c(O)c(Cc5cc(C(C)(C)C)cc(C(C)(C)C)c5)c(O)c(Cc5cc(C(C)(C)C)cc(C(C)(C)C)c5)c4O)=C3O)C(O)=C(Cc3cc(C(C)(C)C)cc(C(C)(C)C)c3)C2=O)cc(C(C)(C)C)c1 ; O=C1C=C(O)/C(=C2/C(=O)C(c3c(O)cc(O)c(C/C=C/c4ccccc4)c3O)=C2O)C(O)=C1C/C=C/c1ccccc1]
or a tautomer thereof.

13. The optical filter of claim 11, wherein the polymer matrix comprises poly(methyl methacrylate) (PMMA).

14. The optical filter of claim 11, wherein the polymer matrix comprises oxygen scavenging agent.

15. The optical filter of claim 11, wherein the filter has a peak absorption of greater than 550 nm.

16. The optical filter of claim 15, wherein the filter has a peak absorbance wavelength of greater than 568 nm.

17. The optical filter of claim 11, or 16, wherein the filter has a full width at half maximum (FWHM) of less than 50 nm.

18. The optical filter of claim 17, wherein the filter has a full width at half maximum (FWHM) of 40-50 nm.

19. A display device comprising the optical filter of claim 11, and an RBG source positioned to allow viewing of the RGB source through the optical filter.

20. The display device of claim 19, wherein the squarylium compound is: 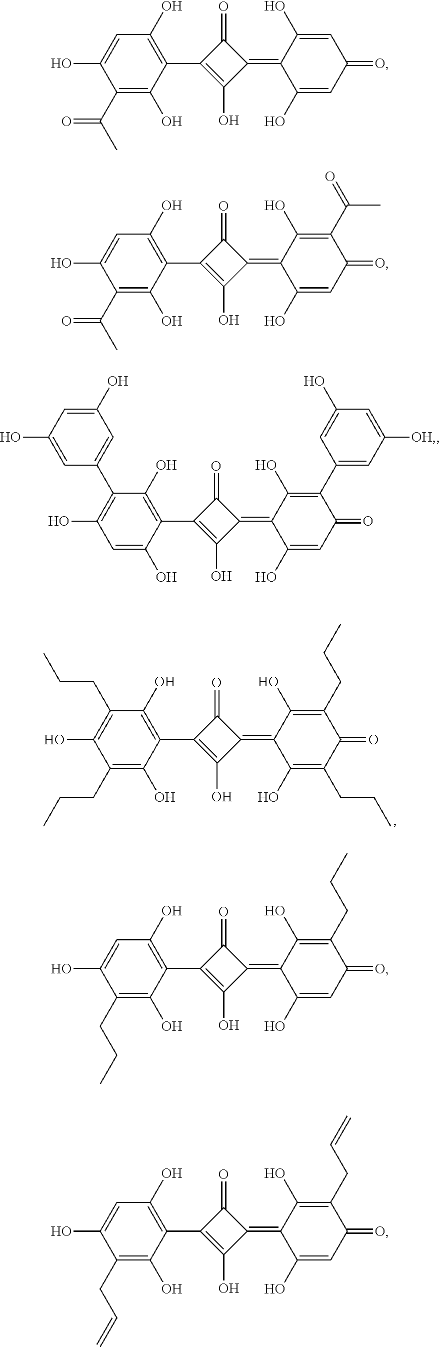 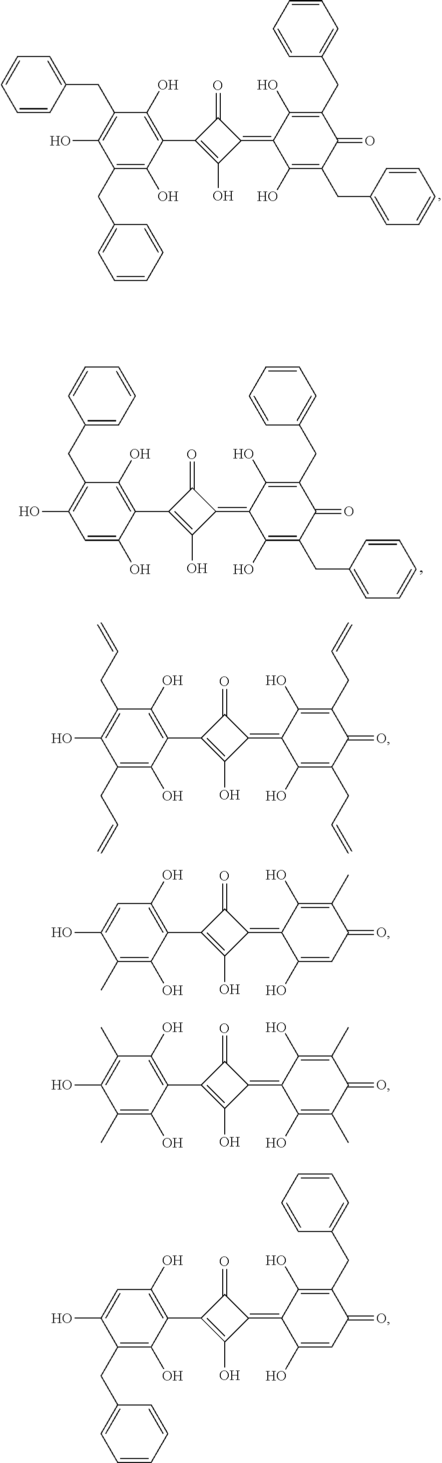 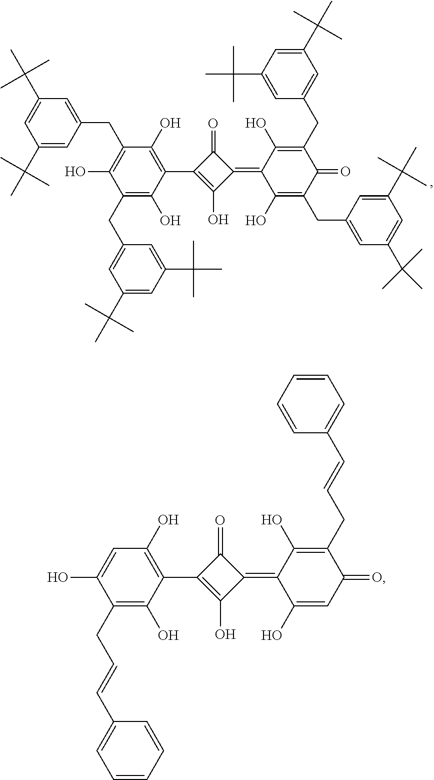 [SMILES: CC(C)(C)c1cc(CC2=C(O)C(=C3C(=O)C(c4c(O)c(Cc5cc(C(C)(C)C)cc(C(C)(C)C)c5)c(O)c(Cc5cc(C(C)(C)C)cc(C(C)(C)C)c5)c4O)=C3O)C(O)=C(Cc3cc(C(C)(C)C)cc(C(C)(C)C)c3)C2=O)cc(C(C)(C)C)c1 ; O=C1C=C(O)/C(=C2/C(=O)C(c3c(O)cc(O)c(C/C=C/c4ccccc4)c3O)=C2O)C(O)=C1C/C=C/c1ccccc1]
or a tautomer thereof.
